SEQUENCE LISTING (10 sequences)

SEQ ID NO: 1 - DNA, 446 bp, Artificial
  partial 16S sequence from E. coli RRSE
ggcggcaggcctaacacatgcaagtcgnacnnnnnnagnnnacagcttgctgtttcgctgacgagtggcggacgggtgagtaatgtctgggaaactgcccgatggagggggataactactggaaacggtagctaataccgcataacgtcgcaagaccaaagagggggaccttagggcctcttgccatcggatgtgcccagatgggattagcttgttggtgaggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgac

SEQ ID NO: 2 - DNA, 468 bp, Artificial
  partial 16S sequence from E. coli RRSG
atcctggctcagattgaacgctggcggcaggcctaacacatgcaagtcgaacggtaacaggaaacagcttgctgtttcgctgacgagtggcggacgggtgagtaatgtctgggaaactgcccgatggagggggataactactggaaacggtagctaataccgcataacgtcgcaagaccaaagagggggaccttagggcctcttgccatcggatgtgcccagatgggattagcttgttggtgaggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgac

SEQ ID NO: 3 - DNA, 474 bp, Artificial
  partial 16S sequence from E. coli RRSH
gtttgatcctggcttagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgacgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagacctcctacgggaggcagcagtggggaatattgcacaatgggcgcaagcctgatgcagccatgccgcgtgtatgaagaaggccttccggttgtaaagtactttcngcgaggaggaaggcattaaggttaataaccttggtgattgac

SEQ ID NO: 4 - DNA, 493 bp, Artificial
  sequence for the “LEICS” strain of H. alvei
tcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgac

SEQ ID NO: 5 - DNA, 496 bp, Artificial
  Australian eae+ “H. alvei” AUS
gcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgac

SEQ ID NO: 6 - DNA, 492 bp, Artificial
  Hafnia alvei type strain HALVEI
caaacttttaaattgaagagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgac

SEQ ID NO: 7 - DNA, 492 bp, Artificial
  partial 16S sequence from E. coli RRSA
caaacttttaaattgaagagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccctcgggcctcttgccatcggatgtgcccagatgggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgac

SEQ ID NO: 8 - DNA, 499 bp, Artificial
  partial 16S sequence from E. coli RRSB
tgagcatcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgac

SEQ ID NO: 9 - DNA, 493 bp, Artificial
  partial 16S sequence from E. coli RRSC
tcaaacttttaaattgaagagtttgatcatggctcagattgaacgctggcggcaggcctaacacatgcaagtcgaacggtaacaggaagcagcttgctgcttcgctgacgagtggcggacgggtgagtaatgtctgggaagctgcctgatggagggggataactactggaaacggtagctaataccgcataatgtcgcaagaccaaagagggggaccttcgggcctcttgccatcggatgtgcccagatgggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgac

SEQ ID NO: 10 - DNA, 499 bp, Artificial; identical to SEQ ID NO: 8
  partial 16S sequence from E. coli RRSD

(19) United States
(12) Patent Application Publication
(10) Pub. No.: US 2010/0099132 A1
(43) Pub. Date: Apr. 22, 2010

(54) BACTERIAL GROWTH INDUCER

(75) Inventors: Primrose Pamela Elaine Freestone, Leicester (GB); Richard David Haigh, Leicester (GB)

Correspondence Address: NIXON PEABODY LLP - PATENT GROUP, 1100 CLINTON SQUARE, ROCHESTER, NY, 14604, US

(73) Assignee: UNIVERSITY OF LEICESTER, Leicester, Leicestershire, SK (GB)

(21) Appl. No.: 12/524,699

(22) PCT Filed: Jan. 28, 2008
(86) PCT No.: PCT/GB2008/050053

§ 371 Date: Jul. 27, 2009

(30) Foreign Application Priority Data
Jan. 26, 2007 (GB) 0701517.5

(51) Int. Cl. C12Q 1/02; C12N 1/20

(52) U.S. Cl. 435/29; 435/252.1

(57) ABSTRACT

The present invention relates to methods of preparing bacterial growth inducers, and in particular to novel bacterial growth inducers/resuscitators prepared by such methods. The invention extends to various applications of such growth inducers, for example in clinical and environmental diagnostics, in reviving not immediately culturable (NIC) bacteria, and in the analyses of microbial populations in blood, food and soil samples.

The present invention relates to methods of preparing bacterial growth inducers, and in particular to novel bacterial growth inducers/resuscitators prepared by such methods. The invention extends to various applications of such growth inducers, for example, in clinical and environmental diagnostics, in reviving not immediately culturable (NIC) bacteria, and in the analyses of microbial populations in blood, food and soil samples.

Bacteria are known to produce various low molecular weight signaling molecules called autoinducers (AI), which are believed to play an important role in the development of bacterial infections in animals and plants. By definition, autoinducers induce or stimulate their own synthesis. Autoinducers may determine whether or not an initial bacterial infection often involving very small numbers of bacterial cells will succumb to the many defence systems of a host organism, or whether such defences are overcome, such that bacterial growth and subsequent infection can occur.

WO 98/53047 describes a purported bacterial autoinducer, referred to by the trade name Bacxell™, which is produced exclusively in a complex, serum-supplemented media (serum-SAPI). A three-step protocol was developed, which enabled purification of the serum-SAPI-produced autoinducer to near homogeneity. Enteric bacterial species, such as E. coli, Salmonella enterica, or Hafnia spp. were suggested as host organisms for producing the autoinducer. Production of the autoinducer was induced by factors which allow the producer bacterial organism to grow from a low cellular inoculum (10²CFU/ml) to the maximum level of growth this media will support, ie about 2×10⁸ CFU/ml.

The autoinducer (Bacxell™) was shown to have utility in a range of applications, for example, in reviving “not immediately culturable” (NIC) bacteria, “viable but non-culturable cells” (VBNCs) or “active but non-culturable” (ABNC), which all have the same meaning, in the analyses of microbial populations in food and soil samples, and in improving recombinant protein expression in fermentations.

Multiple attempts were carried out in the late 1990s to elucidate the structure of Bacxell™, principally using various forms of Mass Spectroscopy. However, the complexity of the serum-based media required for its synthesis, and the apparent instability of the purified Bacxell™ molecule to most of the solvents used in mass spectroscopic analysis, resulted in inconclusive results. Development work on Bacxell™ also slowed significantly because of the complexity and cost of the complex media used in its preparation, the fact that the serum used was bovine-derived, which stimulated BSE fears, and due to the timescale and complexity of the preparation and purification protocols.

Inducers of bacterial growth and autoinducer activity principally include catecholamines, such as norepinephrine (NE), as well as the autoinducer itself. WO 98/53047 describes the use of norepinephrine to stimulate bacterial growth and production of the autoinducer, and is therefore referred to as the NE-induced AI, or NE−AI. The highest concentrations of Bacxell™ were achieved by growing pathogenic E. coli strains, such as E. coli O157:H7, in complex serum-SAPI media, and using NE as the inducer. However, there are significant problems associated with respect to using serum-SAPI media, and using NE, including cost and safety implications. For example, production of Bacxell™ requires a specialised atmosphere (static growth using a humidified 5% CO₂ incubator), which is time-consuming and costly. In addition, while making 5-10 times more autoinducer than enteropathogenic E. coli, and at least 20 times more autoinducer than non-pathogenic K12 strains, the E. coli strain O157:H7 is a containment level 3 pathogen that has an infective dose in mammals of less than 10 CFU. In addition, this restricted-use pathogen has been re-classified. A publication by Lyte and Co-workers (1996) (J Lab Clin Med 128:392, 1996) has reported that E. coli O157:H7 becomes significantly more virulent when cultured under the conditions which are required for autoinducer synthesis, secreting over 100-fold more of the shiga toxin protein which is responsible for haemolytic uraemic syndrome when grown in serum-SAPI media in the presence of NE. Therefore, optimal Bacxell™ production using E. coli O157:H7 cannot currently be performed without highly specialised containment facilities. In addition, the efficient microbiological disposal of waste cultures is also a considerable safety and cost issue with use of E. coli O157:H7. Also, the serum-SAPI media, which is essential for the production of the Bacxell™ autoinducer, contains 30% (v/v) adult bovine serum, which in addition to being an expensive media component, is on occasions, variable in quality, and is subject to occasional microbiological contamination at source. Furthermore, as with any un-fractionated animal fluids, such as blood or serum, it carries with it a small, but real, infection risk.

Further problems associated with producing Bacxell™ are that initial extraction of Bacxell™ from culture media requires prior lyophilisation of the serum-SAPI culture supernatant, and this can take a week to complete. Re-constitution of the lyophilised Bacxell™ serum-SAPI supernatant is slow (due to protein re-solubilisation) and potentially hazardous due to the presence of bacterial toxins. In addition, Bacxell™ production in serum-based media results in a 30% serum waste that still possesses a growth-stimulating activity, due to the low molecular NE−AI binding with very high affinity to serum proteins. This serum-bound NE−AI must be disposed of appropriately, since it is able to enhance growth and virulence of pathogenic bacteria. Disposal of waste NE−AI supernatants is complicated by the serum components stabilising the NE−AI activity rendering it resistant to most disposal procedures, including heat, chlorine or phenolic disinfection and acidification. Three days of treatment with 2M KOH has been found to be effective at inactivating the NE−AI, although there are considerable cost, safety and environmental issues in the regular disposal of large volumes of caustic protein slurries. Apart from the safety issues of disposing of NE−AI waste, binding of the NE−AI to a high molecular weight serum protein means that only about 50% of the NE−AI activity of a Bacxell™ preparation is in a form that can be readily removed from the serum-SAPI culture supernatant. Hence, it will be appreciated that use of the complex media for producing the NE−AI autoinducer compound is inefficient, time-consuming, and expensive.

It is therefore an object of the present invention to obviate or mitigate one or more of the problems of the prior art, whether identified herein or elsewhere, to provide improved, safer and economical processes by which to produce a bacterial growth inducer, which is not dependent on the use of complex media (such as serum) or restricted-use pathogens, for its manufacture. It is a further object of the invention to produce novel bacterial growth inducers, which exhibit improved stabilities.

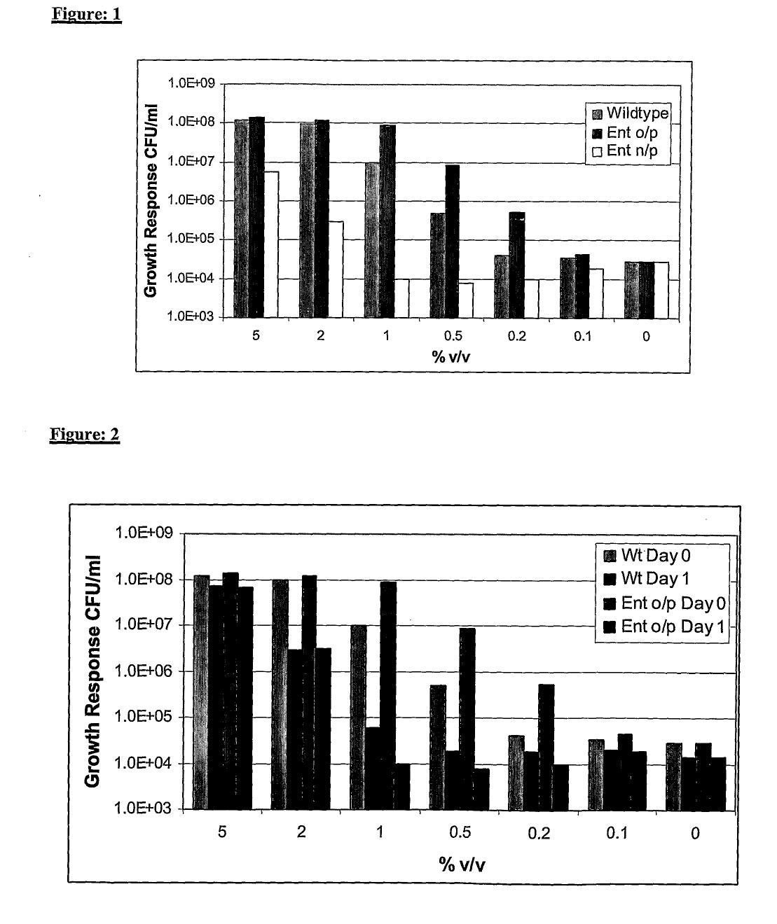

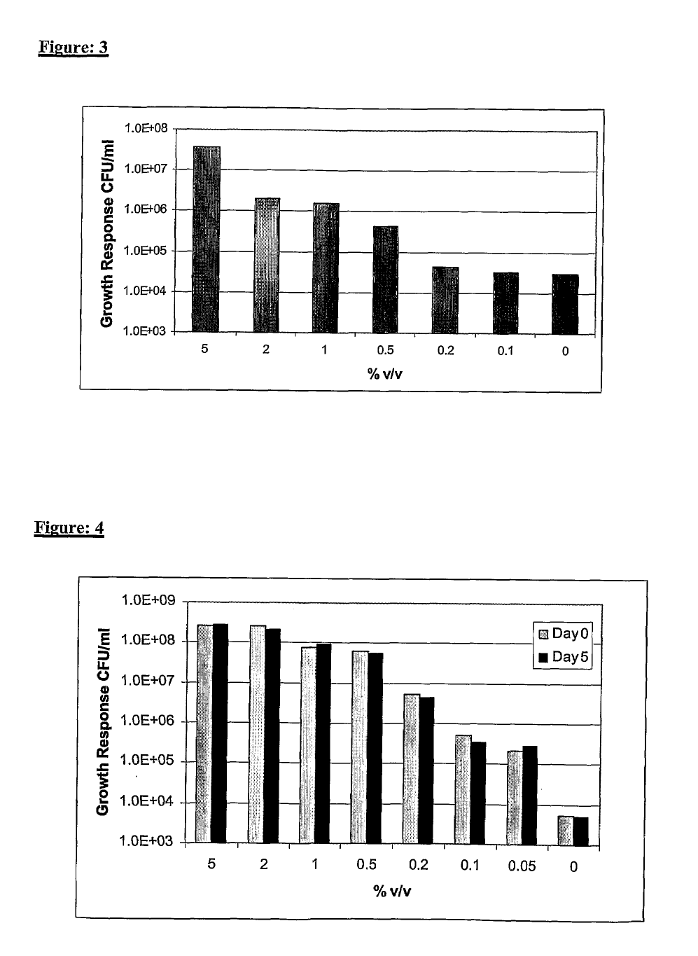

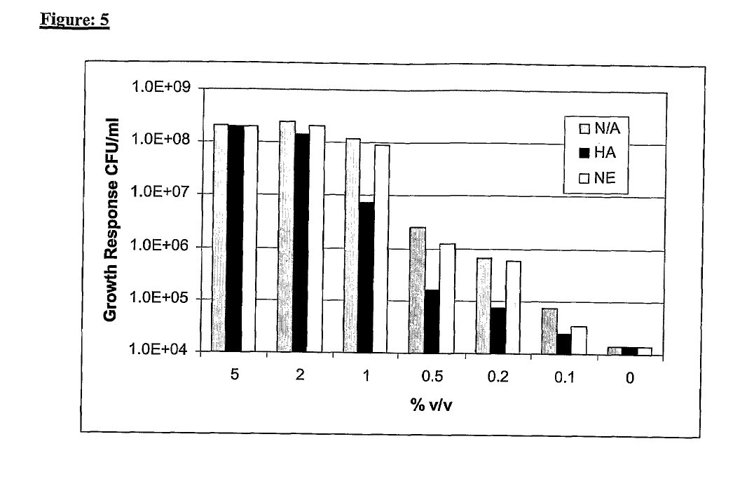

As described in the Examples, following on from their earlier work disclosed in WO 98/53047, the inventors of the present invention carried out various experiments to investigate which micro-organisms produced growth-inducing activities in various media, and also the stability of any activity that was observed. They found that the E. coli and Salmonella strains that were previously employed for synthesis of the prior art NE−AI (ie Bacxell™) were unsuitable for the production of a growth-stimulating activity that possessed the storage stability of the NE−AI when cultured in non-serum supplemented minimal media. The inventors therefore turned their attention to another bacterial species, ie the diarrhoeagenic bacterium, Hafnia alvei. A panel of H. alvei strains was screened for production of growth-stimulating activities in a minimal media, such as M9. Analysis of the M9 supernatants using the methodology described in the legend to FIG. 1, led to the discovery of an H. alvei strain that, when grown in M9 media, exhibited a highly potent growth-stimulating activity, which was also surprisingly stable to storage (as illustrated in FIG. 4). This was most unlike the activity synthesised in M9 under similar conditions by E. coli, as shown in FIGS. 1 and 2.

The bacterial growth-inducing characteristics of the compound produced by H. alvei in minimal media were found to be similar to those described for Bacxell™ However, the most significant difference between the microbial synthesis of the prior art autoinducer, Bacxell™, described in WO 98/53047, and the new compound produced by Hafnia alvei is that Bacxell™ is only produced in serum-supplemented complex SAPI media, with production of the compound being dependent on the addition of a catecholamine, such as noradrenaline (norepinephrine, NE) or the autoinducer (ie Bacxell™) itself. By definition, the autoinducer Bacxell™ is an inducer of its own synthesis. However, in marked contrast, the compound produced by H. alvei in minimal media was produced constitutively when H. alvei is cultured in minimal media such as M9, and is not induced by the addition of either norepinephrine, or itself (FIG. 5). Moreover, also in contrast to the Bacxell™, the compound produced by H. alvei in M9 actually repressed its own synthesis. Hence, the inventors believe that they have isolated a novel bacterial growth-inducer compound by growing H. alvei in M9 minimal media. The compound is able to induce or revive the growth of not immediately culturable (NIC) bacterial cells, and so can be described as an efficient bacterial growth inducer, which exhibits surprising stability to storage (at least two years), but which is not an autoinducer of its own synthesis.

Hence, according to a first aspect of the invention, there is provided a method of producing a bacterial growth inducer, characterized in that the method comprises culturing Hafnia spp. in minimal media, and in that the growth inducer is not an autoinducer.

By the term “autoinducer”, we mean a compound capable of inducing or stimulating its own synthesis.

By the term “bacterial growth inducer”, we mean a compound capable of inducing, stimulating or resuscitating the growth of a bacterium. It is preferred that the bacterial growth inducer is able to induce bacterial growth in substantially bacteriostatic media, such as blood or serum or blood- or serum-supplemented media. The term “bacterial growth inducer” is also taken to encompass a compound which revives, to active growth in both bacteriostatic and non-bacteriostatic media, “not immediately culturable” (NIC) bacteria, “viable but non-culturable cells” (VBNCs) or “active but non-culturable” (ABNC), which all have the same meaning when used herein.

In contrast to the use of complex media for producing the prior art autoinducer, Bacxell™, since the bacterial growth inducer that is produced by the method according to the first aspect of the invention (one embodiment of which is referred to herein as BuGro™) is made constitutively in minimal media without norepinephrine (NE) or serum, the culture media from which it is obtained makes its purification far simpler, and its use in clinical diagnostics, less likely to cause interference with the assay. Furthermore, in contrast to production of Bacxell™, which requires growth of the bacteria in a special atmosphere, the bacterial growth inducer of the invention may be produced under normal atmospheric conditions, which considerably simplifies scale-up production. In addition, the bacterial growth inducer may be directly adsorbed and extracted from culture supernatants using 2×3-hour resin extractions, which takes on average takes 1.5-2 working days to complete, whereas the initial extraction of Bacxell™ requires prior lyophilisation of the serum-SAPI culture supernatant which takes up to week to complete. This demonstrates a clear advantage of producing the bacterial growth inducer of the invention in M9 compared to Bacxell™ in complex serum-based media.

Furthermore, in contrast to Bacxell™ production, because of the simpler composition of the media used to produce the bacterial growth inducer of the invention, it is possible to recover a much higher proportion of the growth inducer from Hafnia spp. culture supernatants. Hence, it will be appreciated from the foregoing that the method according to the invention provides numerous advantages over known methods for producing bacterial growth inducers.

The method according to the invention comprises culturing Hafnia spp. in minimal media. Preferably, the minimal media comprises a glucose-supplemented buffered salt solution. The minimal media may comprise Davis-Mingioli minimal media. However, it is preferred that the minimal media comprises M9 minimal media, which will be known to the skilled technician.

Preferably, the minimal media comprises 0.1-2% (w/v) glucose, more preferably 0.2-1% (w/v) glucose, even more preferably 0.3-0.7% (w/v) glucose, and most preferably about 0.4% (w/v) glucose.

The skilled technician will understand how to prepare M9 media. The preparation of M9 minimal media may comprise (per litre) dissolving 6.0 g of Na₂HPO₄; 3 g of KH₂PO₄; 0.5 g of NaCl; and 1 g NH₄Cl in a litre of nanoQ (18MΩ) water to form a salt solution. Preferably, the solution is sterilized by autoclaving prior to use.

Preferably, 1 ml of sterile 1M MgSO₄ is then added to the salt solution with 10 ml of sterile 40% glucose to give a resulting concentration of 0.4% (w/v) glucose.

The method according to the invention comprises culturing Hafnia spp. in minimal media. The skilled technician will appreciate that Hafnia spp. is a genus of gram-negative facultatively anaerobic rod-shaped bacteria of the family Enterobacteriaceae. A preferred Hafnia species cultured using the method according to the invention is Hafnia alvei. Most preferably, the Hafnia spp. cultured in the method of the first aspect comprises a Hafnia alvei strain isolated by Albert et al (Albert et al., 1991, Infection and Immunity 59:1507-1513; Albert et al., 1992, Journal of Medical Microbiology 37: 310-314). There has been a taxonomic reclassification of diarrhoeagenic Hafnia alvei based on the results of 16S ribosomal RNA gene sequencing and phenotypic analyses which has led to an announcement that diarrhoeagenic Hafnia alvei has been reclassified as a new species: Escherichia albertii (Nuys et al., 2003, International Journal of Systematic and Evolutionary Microbiology 53: 807-810; Hyma et al., 2005, J. Bact. 187:619-628). Hence, the skilled technician will appreciate that Hafnia alvei is synonymous with Escherichia albertii.

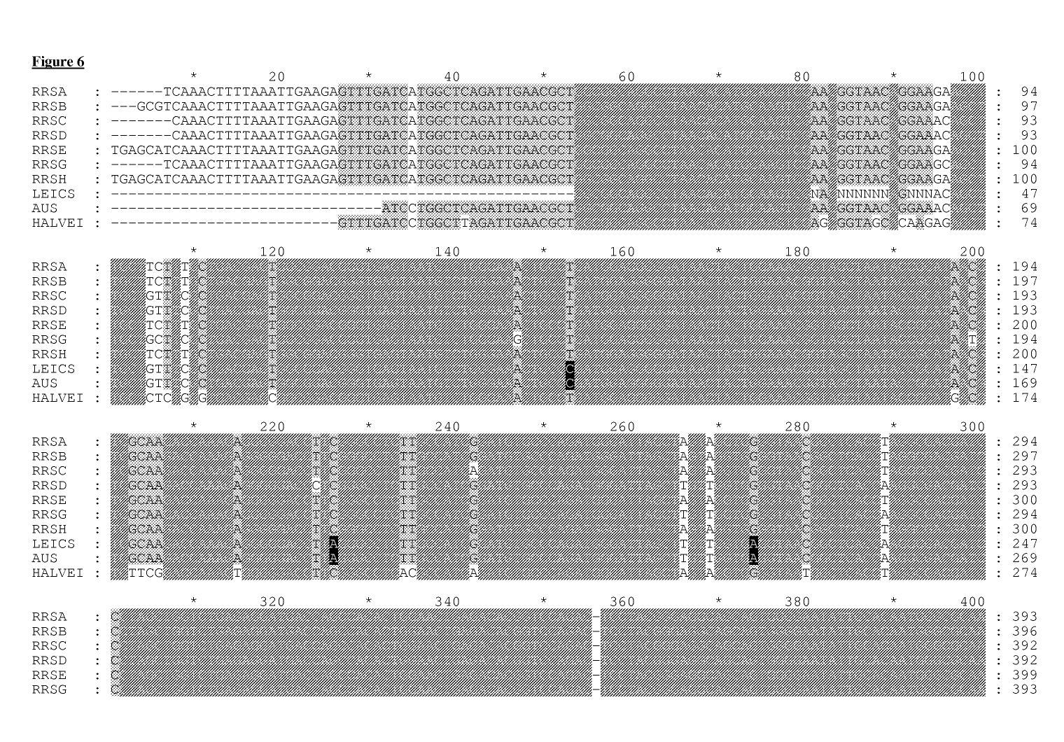

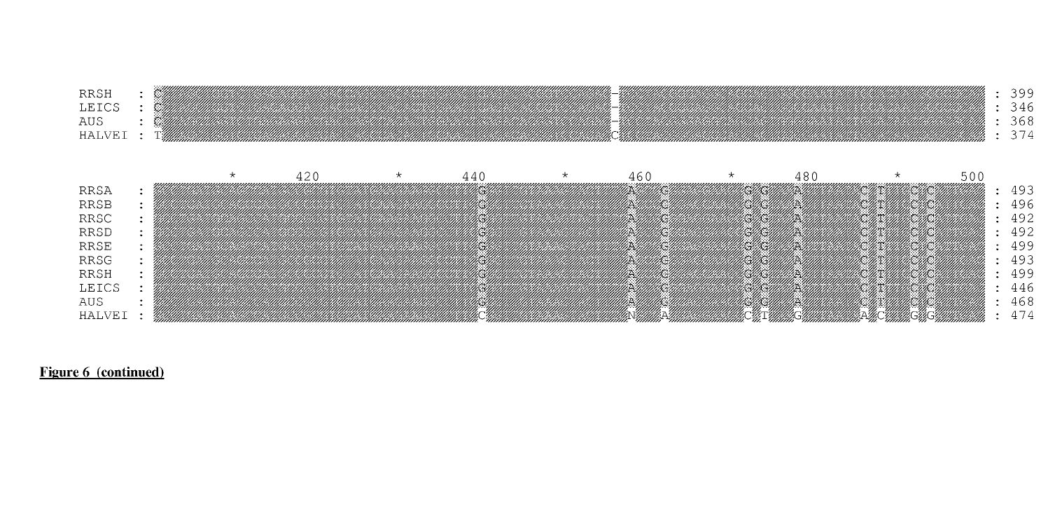

With reference to FIG. 6, the inventors have determined a partial 16S rRNA sequence for their Hafnia alvei strain (denoted LEICS in FIG. 6) used in accordance with the invention. The sequence for the “LEICS” strain of H. alvei (Escherichia albertii) used in accordance with the invention shown in FIG. 6 has the following sequence, denoted SEQ ID No.1:—

SEQ ID No. 1

GGCGGCAGGCCTAACACATGCAAGTCGNACNNNNNNAGNNNACAGCTTGC
TGTTTCGCTGACGAGTGGCGGACGGGTGAGTAATGTCTGGGAAACTGCCC
GATGGAGGGGGATAACTACTGGAAACGGTAGCTAATACCGCATAACGTCG
CAAGACCAAAGAGGGGGACCTTAGGGCCTCTTGCCATCGGATGTGCCCAG
ATGGGATTAGCTTGTTGGTGAGGTAACGGCTCACCAAGGCGACGATCCCT
AGCTGGTCTGAGAGGATGACCAGCCACACTGGAACTGAGACACGGTCCAG
ACTCCTACGGGAGGCAGCAGTGGGGAATATTGCACAATGGGCGCAAGCCT
GATGCAGCCATGCCGCGTGTATGAAGAAGGCCTTCGGGTTGTAAAGTACT
TTCAGCGGGGAGGAAGGGAGTAAAGTTAATACCTTTGCTCATTGAC

Accordingly, it is most preferred that the method according to the invention comprises culturing, in minimal media, a H. alvei strain (ie E. albertii), which may comprise a 16S ribosomal RNA sequence comprising substantially SEQ ID No.1 to produce the non-autoinducer bacterial growth inducer.

Preferably, the culturing step comprises incubating the Hafnia spp. in minimal media at a temperature of about 30° C.-37° C., and preferably about 37° C. The Hafnia spp. may be cultured for at least 5 hours, more preferably at least 10 hours, and even more preferably at least 14 hours, and most preferably for at least 16 hours, or until bacterial growth is maximal. The inventors have found that culturing the Hafnia spp. for about 14-24 hours is suitable to produce the bacterial growth inducer.

Preferably, the method comprises a step of isolating, or purifying, the bacterial growth inducer from the Hafnia spp. growth culture. Preferably, the method comprises collecting a sample from the Hafnia spp. culture containing the bacterial growth inducer. The inventors have found that the bacterial growth inducer is found in significant concentrations in the supernatant of the growth culture. Accordingly, the method may comprise isolating the bacterial growth inducer from a supernatant of the sample or growth culture. The supernatant may be collected from a centrifuged sample containing the growth inducer.

The method may comprise a step of fractionating the sample, and isolating a fraction that corresponds to molecular weights of approximately 800-5000 Daltons. Fractionation may be by means of size exclusion gel filtration. Size exclusion gel filtration may be performed with anion exchange chromatography purification in combination with a suitable buffer (eg approximately 100 mM triethyl ammonium bicarbonate (TEAB), pH7.5). The chromatography may be performed on an anion exchange column, for example using a gradient of 0-1.0 M TEAB. Alternatively, size exclusion gel filtration may be performed using a buffer of approximately 20 mM potassium phosphate containing 150 mM NaCl, pH 7.4, anion exchange purification being performed on an anion exchange column with a NaCl gradient.

The method may comprise an additional step of concentrating the sample prior to the fractionation step. For example, concentration may be achieved by means of ultrafiltration. Alternatively, concentration may be carried out by means of lyophilisation, filtration, or a combination thereof. Hence, size exclusion separation of the bacterial growth inducer may be performed on the sample using preparative ultrafiltration with a molecular weight cut-off (MWCO) that is greater than that of the growth inducer, for example about 1500 Da.

Hence, it is preferred that the method comprises eluting the isolated fraction on an anion-exchange chromatographic column, and selecting a sub-fraction containing the bacterial growth inducer. The method preferably comprises an additional step of performing gel filtration chromatography upon the fraction containing the growth inducer selected in order to isolate the inducer therefrom. Other conditions for performing anion-exchange purification and concentration of the sample will be readily apparent to the skilled technician, particularly with regard to the highly distinctive physical characteristics of the bacterial growth inducer according to the invention.

The inventors believe that the method according to the invention produces a novel compound, which exhibits bacterial growth-inducing properties. Hence, the invention also provides a bacterial growth inducer, which is isolated, and preferably, purified, using the method according to the first aspect.

Therefore, according to a second aspect of the invention, there is provided a bacterial growth inducer, characterized in that the inducer is prepared by, or obtainable by, culturing Hafnia spp. in minimal media, and in that the growth inducer is not an autoinducer.

The inventors have shown that the bacterial growth inducer according to the second aspect that is produced by Hafnia spp. has bacterial growth-inducing activity that is similar to that as described for the prior art autoinducer, Bacxell™ However, in contrast to Bacxell™, the compound according to the second aspect is produced constitutively when Hafnia alvei is cultured in minimal media, and is not induced by the addition of either norepinephrine, or itself. Also, surprisingly, the compound according to the second aspect represses its own synthesis. Hence, the growth inducer of the second aspect is not an autoinducer.

The inventors were also surprised to observe that the bacterial growth inducer according to the second aspect has a number of differences to the prior art autoinducer (Bacxell™) disclosed in WO 98/53047. Table 2 summarises the key similarities between the prior art autoinducer Bacxell™ and the novel bacterial growth inducer of the invention.

For example, the bacterial growth inducer according to the second aspect of the invention may be characterized in that it has absorption maxima at about 280 nm. The growth inducer may fluoresce blue under UV light. Preferably, the growth inducer is substantially electronegative. Preferably, the growth inducer is substantially soluble in water. Preferably, the growth inducer is substantially insoluble in organic solvents such as ethanol, methanol or chloroform. Preferably, the growth inducer is substantially stable to lyophilisation. Preferably, the growth inducer is substantially stable to heating (for example to temperatures over 100° C.). Preferably, the growth inducer is substantially stable to storage. Preferably, the growth inducer is substantially stable to digestion by ribonuclease, deoxyribonuclease, protease, phosphatase, or phosphodiesterase. Preferably, the growth inducer is substantially unstable to acidic pH (between pH 1-6), but more stable in neutral (pH7) or alkaline conditions (pH 8-12).

The bacterial growth inducer according to the second aspect of the invention may be characterized in that it has a molecular weight of about 800-5000 Daltons.

Preferably, the growth inducer has a molecular weight of between about 900 and 2000 Daltons, more preferably between about 1000 and 1500Da, and most preferably between about 1050 and 1200 Da.

Preferably, the growth inducer exhibits siderophore activity.

By the term “siderophore”, we mean an iron-chelating compound secreted by micro-organisms grown under iron-limited conditions which is used to facilitate cellular iron transport. Iron (Fe³⁺) ions have a very low solubility at neutral pH and therefore cannot be utilized by organisms. However, siderophores dissolve these ions as soluble Fe³⁺ complexes that can be taken up by microbial active transport mechanisms. The siderophore enterobactin is a tri-catechol derivative of a cyclic tri-serine lactone. Studies of the chemistry, regulation, synthesis, recognition, and transport of enterobactin make it one of the best understood of the siderophore-mediated iron uptake systems. However, unlike the NE−AI, Bacxell™, which has no siderophore activity as demonstrated in Freestone et al (2003) Microbiol. Lett. 222: 39-43, the inducer of the second aspect can effectively substitute for the siderophore enterobactin in test assays, and therefore has been shown to exhibit siderophore activity. In view of this surprising observation, the inventors believe that enterobactin genes may be involved in the synthesis of the growth inducer.

Preferably, the growth inducer is synthesized in Hafnia spp., and more preferably H. alvei (E. albertii) only. Preferably, the growth inducer is produced constitutively. Preferably, addition of the growth inducer according to the second aspect to a culture of Hafnia alvei in minimal media represses its own synthesis. Preferably, addition of norepinephrine to a culture of Hafnia alvei in minimal media does not induce synthesis of the bacterial growth inducer according to the second aspect.

Preferably, the bacterial growth inducer compound according to the second aspect is characterized in that the production of the growth inducer is repressed under conditions of iron excess, as shown in the examples. Preferably, the growth inducer is capable of stimulating bacterial growth and/or virulence factor expression. Preferably, the growth inducer is capable of reviving viable but non-culturable bacteria, and also stressed (for example, heat-stressed) bacteria.

Preferably, the growth inducer according to the invention exhibits any or all of above properties.

Preferably, the bacterial growth inducer compound according to the second aspect of the invention is characterized in that:—
(i) it has a molecular weight of about 800-5000 Daltons;
(ii) it exhibits siderophore activity;
(iii) it is fluorescent;
(iv) it is produced constitutively;
(v) addition of the growth inducer to a culture of Hafnia alvei in M9 represses its own synthesis;
(vi) addition of norepinephrine inducer to a culture of Hafnia alvei in M9 does not induce synthesis of the bacterial growth inducer; and/or
(vii) the production of the growth inducer is repressed under conditions of iron excess.

The bacterial growth inducer according to the invention may be used to induce bacterial growth, either of bacteria of the species from which the growth inducer was obtained (ie Hafnia spp.), or of other species.

For example, the bacterial growth inducer may be capable of inducing the growth of a Gram-positive or Gram-negative bacterium. For example, bacteria whose growth may be induced by the growth inducer of the invention include Firmicutes, which may be Bacilli or Clostridia, for example Clostridium botulinum. Further examples of bacteria with which the growth inducer may be effective may include Bacillales, such as Bacillus subtilis.

The inducer may be effective at inducing growth of Staphylococcus, for example, Staphylococcus aureus. The inducer may be particularly effective at inducing the growth of MRSA (methicillin-resistant S. aureus) strains. Additional Bacillales with which the inducer may be effective include Streptococci, for example, Streptococcus pyogenes or Streptococcus pneumoniae.

Further examples of bacteria with which the inducer are effective may include Pseudomonadales, such as Pseudomonas aeruginosa. The inducer may also include growth in Xanthomonas spp. Further examples of bacteria against with which the inducer is effective may include Gammaproteobacteria, which may be selected from a group consisting of Enterobacteriales, Proteus, Serratia, Pasteurellales, and Vibrionales.

Suitable Enterobacteriales with which the inducer is effective include Escherichia spp., such as E. coli. Other suitable Enterobacteriales with which the inducer is effective include Salmonella spp., such as S. enterica or S. bongori. Other suitable Enterobacteriales with which the inducer is effective include Shigella spp., such as S. sonnei, S. flexneri, S. dysenteriae, or S. boydii. Further suitable Enterobacteriales with which the inducer is effective include Yersinia spp., such as Y. pestis, Y. entercolitica, or Y. pseudotuberculosis. Further suitable Enterobacteriales with which the inducer is effective include Klebsiella spp., such as K. oxytoca or K. pneumoniae. Further suitable Enterobacteriales with which the inducer is effective include Erwinia spp., such as E. amylovora or E. carotovora.

Examples of Proteus with which the inducer is effective include Proteus mirabilis. Examples of suitable Serratia include Serratia marcescens. Examples of Pasteurellales include Haemophilus influenzae.

Further examples of bacteria with which the inducer according to the invention is effective may include Proteobacteria, including Neisseriales, for example, Neisseria gonorrhoeae. Further examples of bacteria with which the inducer is effective may include Delta/epsilon subdivided Proteobacteria, including Campylobacteriales, for example Helicobacter pylori. Further examples of bacteria with which the inducer is effective may include Actinobacteria, for example Mycobacterium tuberculosis and Nocardia asteroides. The inducer may also be effective with Vibrio spp., such as V. cholerae, V. parahaemolyticus, or V. vulnificus.

It is especially preferred that the bacterial growth inducer may be used to induce growth of not immediately culturable (NIC) bacteria. Such bacteria may be environmentally-stressed bacteria, such as those that would be found in environmental- or clinical-diagnostic samples. It will be appreciated that the growth inducer has a wide range of possible uses, including anything in which the growth of a bacterium or the production of a desired molecule from a bacterial cell, is to be stimulated or assayed.

Hence, in a third aspect, there is provided use of the bacterial growth inducer according to the second aspect for inducing growth of bacteria in a sample.

Various applications for the growth inducer are described in Examples 10 to 14. For example, the growth inducer may be used in fermentation processes, in culture media for diagnostic and environmental monitoring, detection of bacteria in a sample, or in the drug discovery process in order to find agents which will inhibit bacterial growth inducer-mediated bacterial stimulation. In fermentation processes, the growth inducer may be used to stimulate starting cultures, or to shorten and synchronise lag phases. It may also be used in fermentation processes to stimulate the production of a secondary metabolite, such as an antibiotic, or chemical for biological screening. The growth inducer may also be used in culture media to shorten turn-around times or to assay viable, but non-culturable organisms. Other uses of the bacterial growth inducer according to the invention will be readily apparent to the skilled technician.

In a fourth aspect, there is provided a method of inducing growth of bacteria in a sample, the method comprising contacting a sample containing bacteria with the bacterial growth inducer according to the second aspect, and incubating said sample under conditions suitable for inducing growth of the bacteria.

In a fifth aspect, there is provided a bacterial growth induction kit for inducing growth of bacteria in a sample, the kit comprising the bacterial growth inducer according to the second aspect, and optionally instructions for use.

The skilled technician will appreciate that the method or use of the kit comprises an initial step of obtaining a sample containing bacteria (or which is believed to contain bacteria), followed by a step of contacting the sample with the bacterial growth inducer. The inducer may be combined with the usual culture media that would normally be used to analyse the sample. Alternatively, the inducer may be supplied separately in which case it will be added to the culture media prior to the incubation step. The inducer may be provided in a suitable container.

The resultant mixture may then be incubated under suitable culture conditions to enable the inducer to act upon the bacteria, and induce growth. It will be appreciated that “suitable culture conditions” means for sufficient time and at the required temperature for the bacterial population in the sample to grow, and this will be dependent on the specific bacterial population that is in the sample. Accordingly, the skilled technician will appreciate that the suitable culture conditions will need to be chosen depending on the type of sample being tested. By way of example, the incubating may be carried out at a temperature of between about 20° C. and 40° C., preferably between about 24° C. and 40° C., and more preferably between about 30° C. and 40° C. (most preferably about 37° C.), for at least 5, 10, 12, 14, 16, 18, 20, 22, or at least 24 hours. In some embodiments, the incubating step may require at least 48 hours, or even at least 72, or even at least 96 hours, for the growth inducer to sufficiently induce bacterial growth.

Once the inducer compound induces the bacteria to grow, the sample is maintained at optimum culturing conditions to enable the bacterial population to be revived. The method preferably comprises a detection step for detecting the growing bacteria. For example, the sample may be plated out onto suitable media, and the number of bacterial colonies may be counted.

The sample containing the bacteria may be any sample in which a population of bacteria is believed to exist, and for which it is required to induce, revive or accelerate their growth. The bacteria may comprise not immediately culturable (NIC) bacteria, which may otherwise go undiscovered in the sample, and thereby avoid detection. For example, the sample may be a blood sample, a food sample or a soil sample, each of which are known to frequently contain low levels of bacteria which may not be easily culturable using routine cell culture or diagnostic techniques. Therefore, it will be appreciated that the addition of the growth inducer of the second aspect to a sample containing such bacteria will induce their growth and increase the chances of their detection.

The inventors believe that the method according to the fourth aspect and kit of the fifth aspect may be especially important in the field of blood diagnostics.

Therefore, in a sixth aspect, there is provided a method of inducing growth of bacteria in a blood sample, the method comprising contacting a blood sample containing bacteria with the bacterial growth inducer according to the second aspect, and incubating said sample under conditions suitable for inducing growth of the bacteria.

In a seventh aspect, there is provided a blood bacterial population detection kit for inducing growth of bacteria in a blood sample, the kit comprising the bacterial growth inducer according to the second aspect, and optionally instructions for use.

Examples 10 to 12 describe preferred embodiments for the method according to the sixth aspect or the kit of the seventh aspect. The skilled technician will appreciate that the method or use of the kit comprises initially obtaining a blood sample containing bacteria, which may be not immediately culturable (NIC) bacteria, contacting the blood sample with the bacterial growth inducer, and then culturing the mixture under suitable culture conditions (for sufficient time at the required temperature) to enable the inducer to act. Once the inducer compound induces the bacteria to grow, the sample may be plated out onto suitable media, and the number of bacterial colonies counted. Hence, it is possible to diagnose the population of not immediately culturable bacteria present in the blood sample. Hence, the method and kit may be used to provide an early warning of a potential infection of pathogenic bacteria in blood, and so a treatment regime may be initiated in advance of full-blown bacterial infection. The inventors have surprisingly found that the growth inducer is even capable of inducing growth of antibiotic-damaged bacteria. Accordingly, in circumstances where a patient suffering from a bacterial infection may have undergone treatment with a course of antibiotics, it should still be possible to assess the extent of the bacterial infection post-treatment by using the kit of the sixth aspect.

The inventors believe that the method according to the fourth aspect and kit of the fifth aspect may also be especially useful in the field of diagnosing the microbial population of a food sample, for example, for detecting Salmonella.

Therefore, in an eighth aspect, there is provided a method of inducing growth of bacteria in a food sample, the method comprising contacting a food sample containing bacteria with the bacterial growth inducer according to the second aspect, and incubating said sample under conditions suitable for inducing growth of the bacteria.

In a ninth aspect, there is provided a food bacterial population detection kit for inducing growth of bacteria in a food sample, the kit comprising the bacterial growth inducer according to the second aspect, and optionally instructions for use.

Example 13 describes a preferred embodiment for the method according to the eighth aspect or the kit of the ninth aspect. The skilled technician will appreciate that the method or use of the kit comprises obtaining a food sample (such as dried herbs) containing bacteria, contacting the food sample with the bacterial growth inducer, and culturing the mixture under suitable culture conditions (for sufficient time at the required temperature) to enable the inducer to induce bacterial growth. Hence, it is possible to diagnose the population of not immediately culturable (NIC) bacteria present in the food sample. Example 13 demonstrates that the bactieral species Erwinia, Klebsiella and Xanthomonas may be detected in this manner.

The inventors believe that the method according to the fourth aspect and kit of the fifth aspect may also be especially useful in the field of diagnosing the microbial population of a soil sample.

Therefore, in a tenth aspect, there is provided a method of inducing growth of bacteria in a soil sample, the method comprising contacting a soil sample containing bacteria with the bacterial growth inducer according to the second aspect, and incubating said sample under conditions suitable for inducing growth of the bacteria.

In an eleventh aspect, there is provided a soil bacterial population detection kit for inducing growth of bacteria in a soil sample, the kit comprising the bacterial growth inducer according to the second aspect, and optionally instructions for use.

Example 14 describes a preferred embodiment for the method of the tenth aspect or the kit of the eleventh aspect. The skilled technician will appreciate that the method or use of the kit comprises obtaining a soil containing bacteria, contacting the soil sample with the bacterial growth inducer, and culturing the mixture under suitable culture conditions (for sufficient time at the required temperature) to enable the inducer to induce bacterial growth in the soil. Hence, the method may be used to provide early warning of a potential infection of pathogenic bacteria in the soil.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

FIG. 1 is a bar chart demonstrating that E. coli producers of norepinephrine-induced autoinducer (NE−AI) do not synthesise a stable growth-stimulating compound in M9 minimal media. E. coli E2348/69 strains wild-type (Wt), enterobactin over-producer fes mutant (Ent o/p) and enterobactin non-producer entA mutant (Ent n/p) were inoculated at 2×10⁶ CFU/ml into M9 minimal media supplemented with 0.4% glucose;

FIG. 2 is another bar chart demonstrating that E. coli producers of norepinephrine-induced autoinducer (NE−AI) do not synthesise a stable growth stimulating compound in M9 minimal media. The supernatants from the wild-type (Wt) and enterobactin over-producer (Ent o/p) cultures produced in FIG. 1 were re-assayed as described 24 hrs (Day 1) after storage at 4° C.;

FIG. 3 is a bar chart demonstrating that Salmonella producers of norepinephrine-induced autoinducer (NE−AI) synthesise only low levels of a growth-stimulating compound in M9 minimal media. The values shown are mean values of duplicate plate counts, and variation between individual assays was no more than 5%;

FIG. 4 is a bar chart demonstrating that H. alvei (E. albertii) synthesizes a stable growth-stimulating compound in M9 minimal media. The novel compound is referred to herein as “BuGro™”. E. albertii was inoculated at 2×10⁶ CFU/ml into M9 minimal media supplemented with 0.4% glucose, and growth response activity was measured at Day 0, and 5 days later (Day 5);

FIG. 5 is a bar chart demonstrating that production of the H. alvei growth-stimulating compound is constitutive, and not induced by norepinephrine. H. alvei was inoculated at 2×10⁶ CFU/ml into M9 minimal media supplemented with 0.4% glucose only (N/A), or with 5% culture supernatant from a previous H. alvei M9 culture (approximately 50 units/ml) (HA), or with 100 μM norepinephrine (NE);

FIG. 6 is a sequence alignment of partial 16S rRNA sequences from E. coli (rrsA-H), the Leicester “H. alvei” (Escherichia albertii; LEICS) used in the methods of the invention, the published Australian eae⁺ “H. alvei” (Escherichia albertii; AUS) and a Hafnia alvei type strain (HALVEI). The varying grey backgrounds indicate decreasing degrees of concurrence between the ten 16S rRNA sequences; a large number of differences can be observed between “real” Hafnia alvei sequence and the Escherichia sequences. The Leicester H. alvei (Escherichia albertii) sequence in accordance with the invention and the Australian eae⁺ H. alvei (Escherichia albertii) sequence (Genbank Accession number Z83203) are 100% identical (except for the 14 by where the DNA sequencer failed to resolve: residues 80-95 indicated by N). Both sequences contain three distinguishing nucleotide differences (indicated by white letters on black background) which are not in any of the 7 E. coli 16S rRNA gene sequences, or in H. alvei.

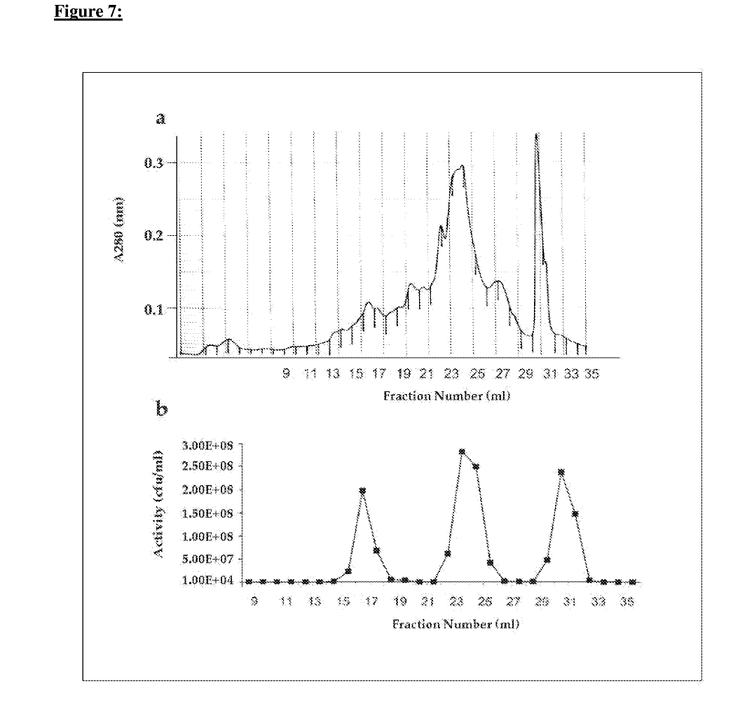

FIG. 7 shows the results of a Superdex pep analytical chromatography column fractionation profile of crude BuGro™. FIG. 7(a) is a UV chromatogram of a typical Superdex pep column fractionation of concentrated crude BuGro™ (the fractionation profile for about 70,000 units of BuGro™ activity is shown); and FIG. 7(b) is a growth-stimulating activity profile for 1 μl of the 1 ml fractions shown in (a), showing the positions of BuGro™ activity peaks 1, 2 and 3;

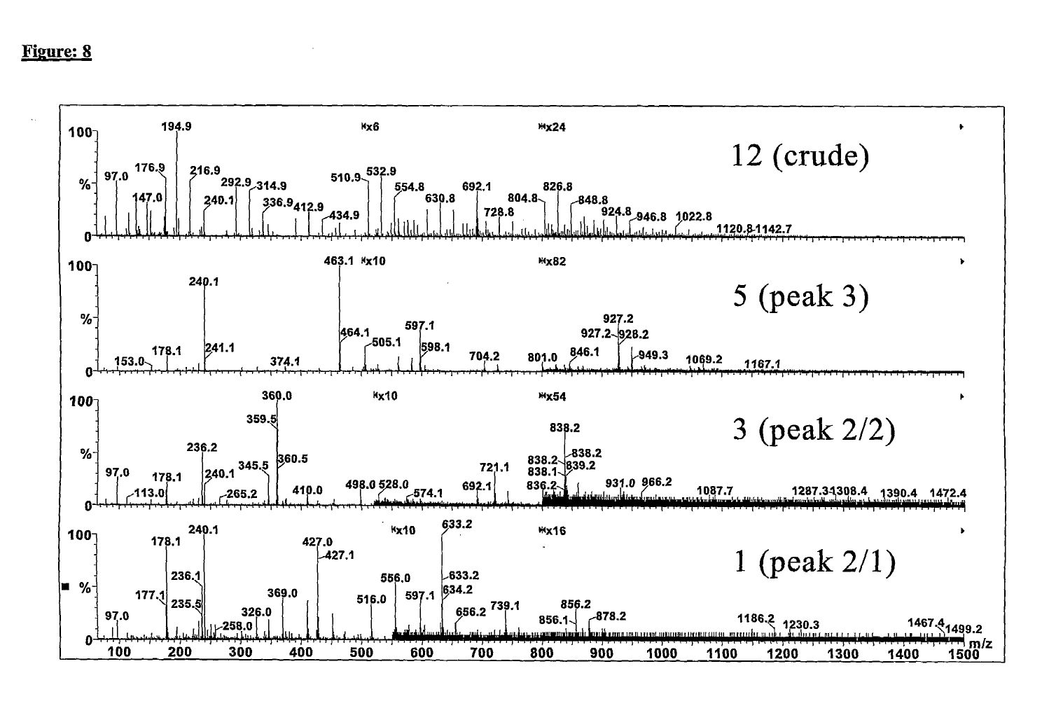

FIG. 8 shows mass spectroscopy analysis of crude BuGro™ and Peaks 2/1, 2/2 and 3. The figures show Electro Spray Ionisation mass spectra (ESI) of BuGro™ peaks 2/1, 2/2, 3, and crude extract, all in the positive ion mode of detection;

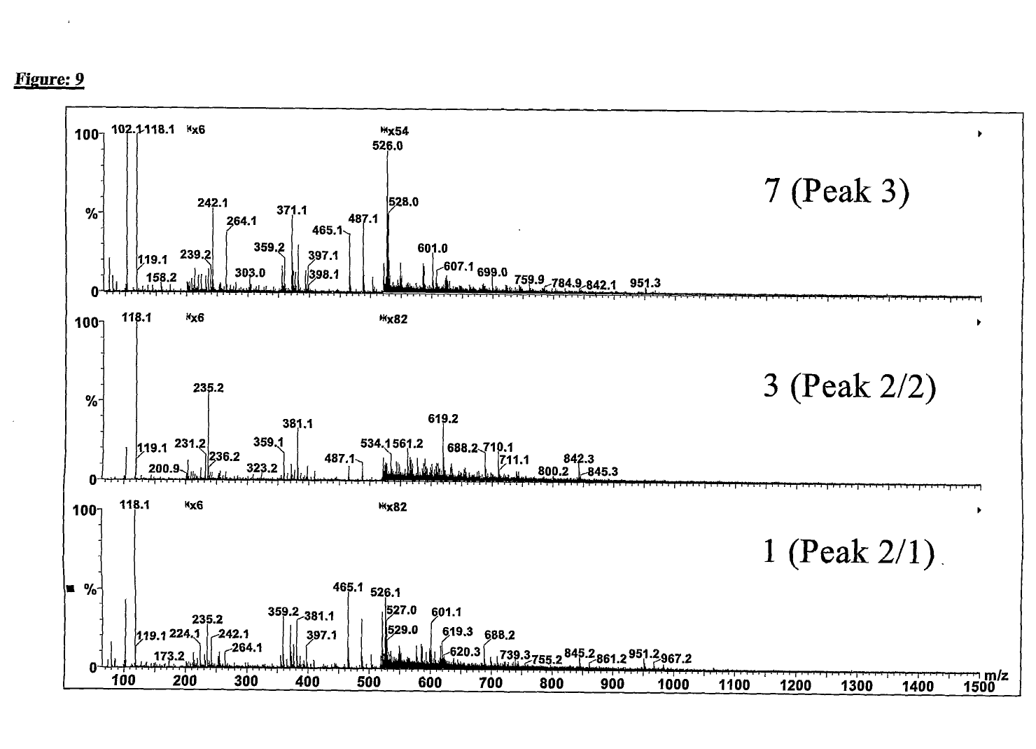

FIG. 9 shows ESI mass spectra of BuGro™ peaks 2/1, 2/2, and 3 in the negative ion mode of detection;

FIG. 10 is a table showing the masses of prominent Peak 2/1 and 2/2 fragmentation products in the negative ion mode. First row—precursor ions; columns—fragmentation product ions of each precursor;

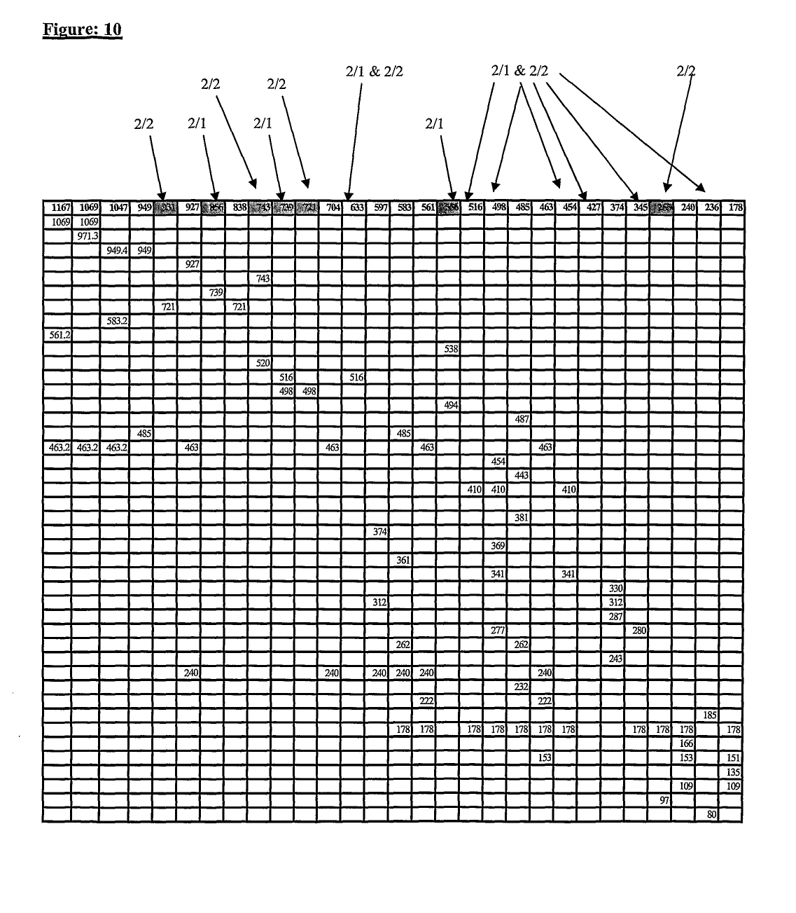

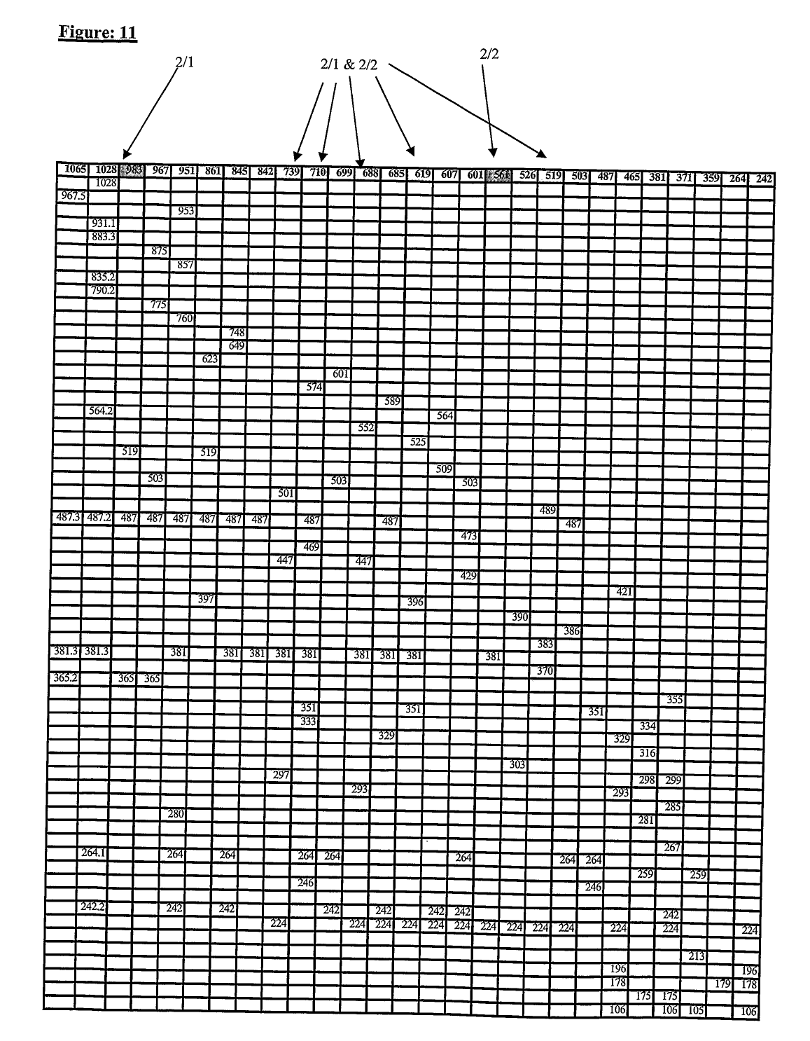

FIG. 11 is a table showing the masses of prominent Peak 2/1 and 2/2 fragmentation products in the positive ion mode. First row, precursor ions; columns—fragmentation product ions of each precursor;

FIG. 12 shows a bar chart illustrating the ability of crude BuGro™ and purified Peaks 1, 2 and 3 to enable 2×10⁸ CFU/ml of an entA enterobactin-deficient mutant strain of E. coli E2348/69 to acquire iron (in the form of ⁵⁵Fe) from ⁵⁵Fe-labelled transferrin. Key: No additions (N/A); 100 units/ml of Peaks 1, 2 and 3 only (P1, P2, P3); 100 units/ml of Peaks 1, 2 and 3 plus 100 μM norepinephrine (P1+NE, P2+NE, P3+NE); 100 μM norepinephrine only (NE); 50 units/ml crude BuGro only (HA); 50 units/ml crude BuGro plus 100 μM NE (HA+NE); enterobactin (from wild-type E. coli) plus NE; 200 units/ml NE−AI plus 100 μM NE (NE−AI+NE);

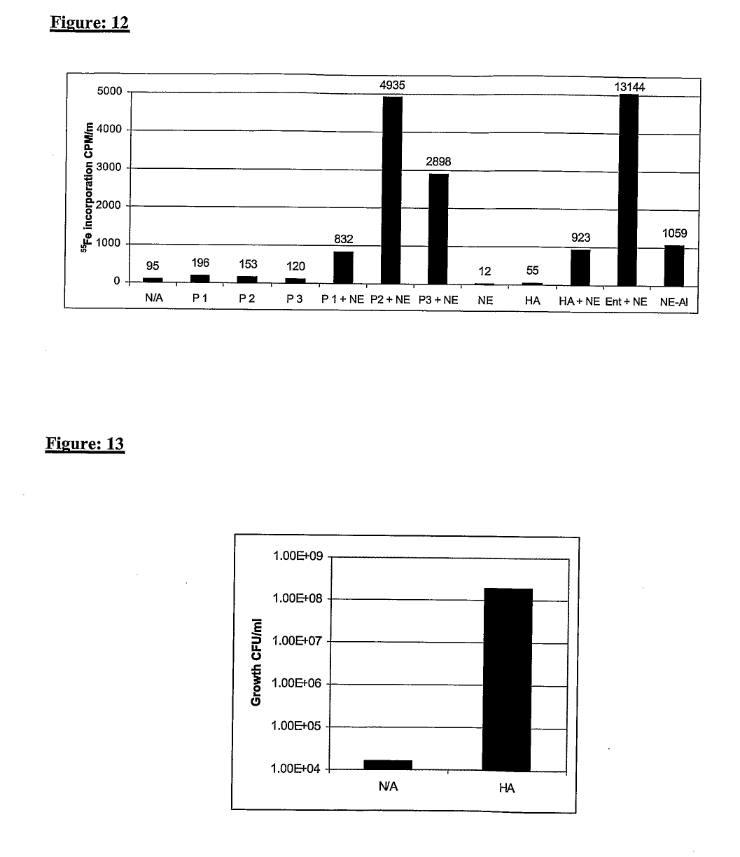

FIG. 13 shows BuGro™ enhancement of growth of bacteria in blood-supplemented media. The figure shows the growth levels of ten E. coli cells inoculated into 10 ml of BacT/ALERT culture media+30% blood with either no additions (N/A) or 50 units/ml BuGro™, and incubated for 14 hours at 37° C.;

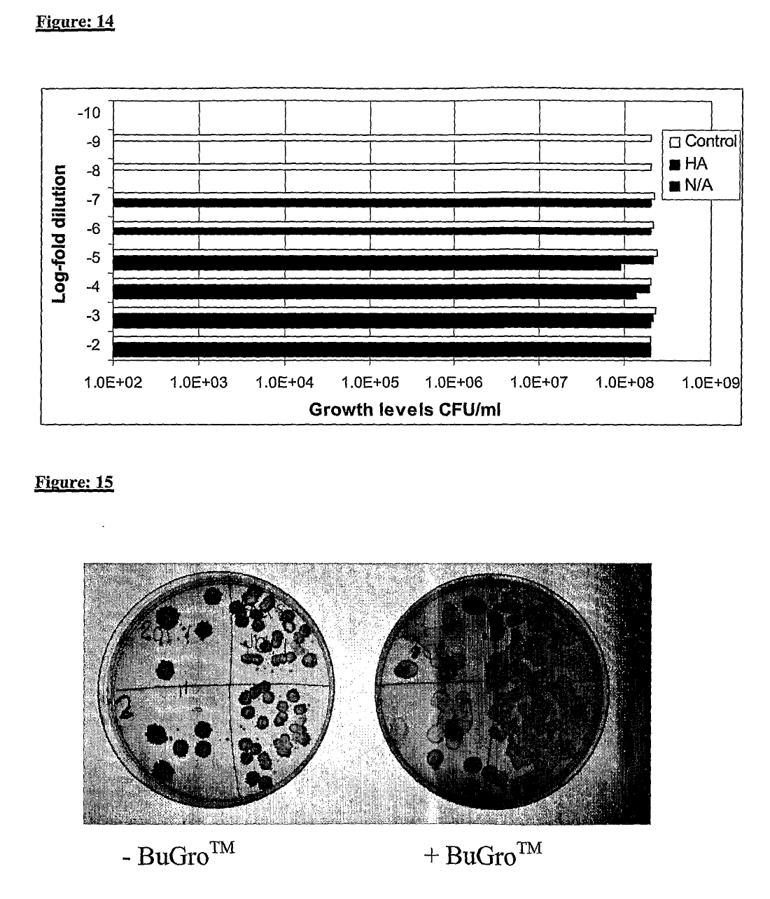

FIG. 14 shows the growth levels of 10⁸ CFU/ml ampicillin-treated E. coli cells serially diluted into BacT/ALERT media (no blood) supplemented with either no additions (N/A) or 50 units/ml BuGro™ (HA) and incubated for 14 hours at 37° C. Growth levels were determined by serial dilution of the cultures and plate counting using Luria agar;

FIG. 15 shows bacterial plates in the presence (+BuGro™) and absence (−BuGro™) of BuGro™ in the analyses of the microbial composition of food samples;

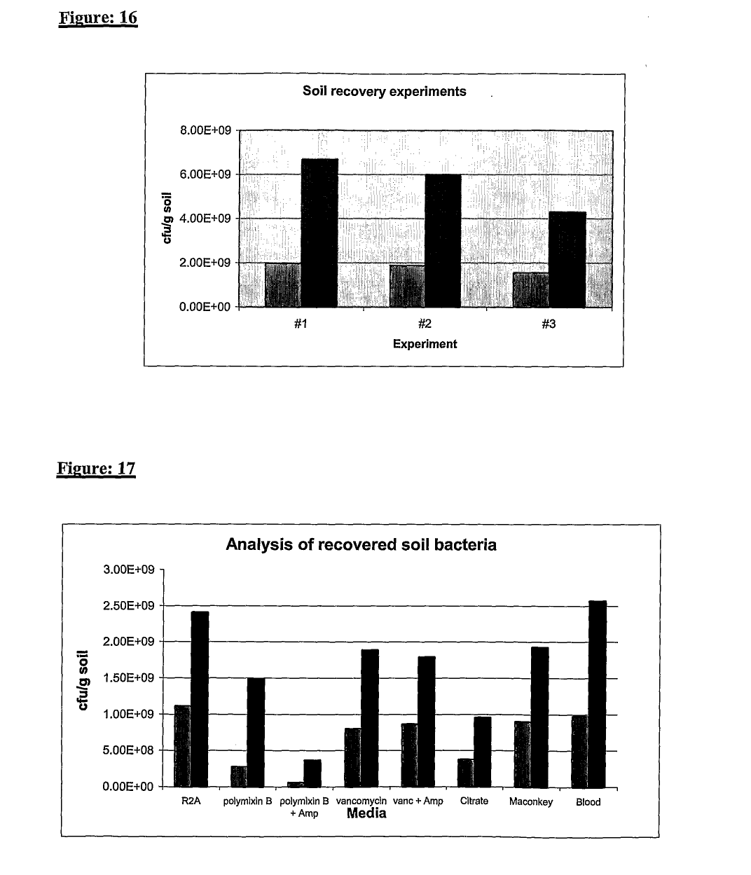

FIG. 16 shows bacterial growth levels in soil recovery experiments. Key: control (ie no additions): light grey bars, and 10 units/ml BuGro™: black bars;

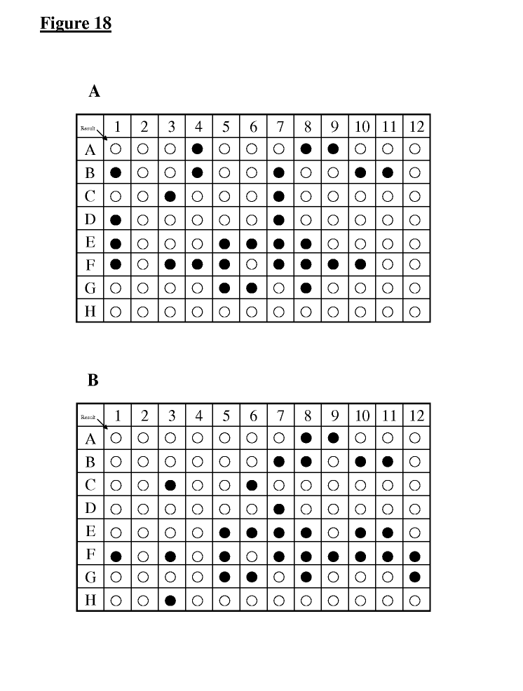

FIG. 17 shows the analysis of recovered soil bacteria. Key: control (ie no additions) light grey bars, 10 units/ml BuGro™, black bars;

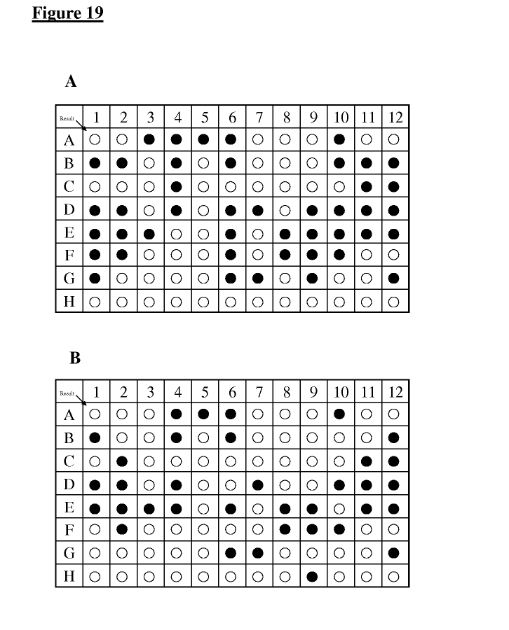

FIG. 18 shows a Biolog™ Microplate metabolic fingerprint of Gram-positive soil bacteria grown in the presence (A) or absence of 10 units/ml of purified BuGro™ (B). The plates shown were incubated at room temperature for 7 days; and

FIG. 19 shows the Biolog™ Microplate metabolic fingerprint of Gram-negative soil bacteria grown in the presence (A) or absence of 10 units/ml of purified BuGro™ (B). The plates shown were incubated at room temperature for 7 days.

### EXAMPLES

Previous work carried out by the inventors of the present invention, which is published in WO 98/53047, concerned the production of an autoinducer called Bacxell™ by growing E. coli in a complex serum-SAPI production media. The inventors of the present invention have now found that the SAPI component of the complex serum-SAPI production media for the Bacxell™ autoinducer when used alone is far too nutritionally limited to support anything but very low levels of bacterial growth. The inventors have also found that the enteropathogenic E. coli strain, E2348/69, is one of the most potent enteric producers of the Bacxell™ autoinducer, and so they carried out analyses to determine whether this strain was able to synthesise growth-stimulating compounds that possessed similar characteristics to the Bacxell™ autoinducer in other media.

### Example 1

### Growth of E. coli in Minimal M9 Media

Preparation of M9 minimal media is as follows: Na₂HPO₄.2H₂O—33.95 g; or Na₂HPO₄ anhydrous—41.50 g; KH₂PO₄—15 g; NaCl—2.5 g; and NH₄Cl—5 g were first weighed out. These chemicals were dissolved in 500 ml nanoQ (18MΩ) H₂O, and then diluted 1:10 with nanoQ (18MΩ) H₂O, and autoclaved to sterilize. 40% glucose was then aseptically added to a resulting concentration of 0.4% (w/v) glucose. The M9 minimal media supplemented with 0.4% (w/v) glucose was then separately inoculated at 2×10⁶ CFU/ml with three different strains of E. coli, ie E. coli E2348/69 wild-type (Wt), enterobactin over-producer fes mutant (Ent o/p) and enterobactin non-producer entA mutant (Ent n/p).

Enterobactin is a siderophore, ie an iron-chelating compound, secreted by the wild-type and overproducer (fes) E. coli strains, but not the entA mutant strain. The cultures were incubated at 37° C. in a rotatory shaking incubator for 18 hours, achieving similar final cell densities of 2.26, 2.21 and 1.91×10⁸ CFU/ml respectively. Bacteria were removed by centrifugation at 10,000 g for 15 minutes, and the resulting culture supernatant was sterilised by passage through a 0.2 micron stericup filter. The sterile supernatant was assayed at a range of dilutions of the three cultures to determine the effect it had on the growth response against a test bacterial strain E. coli E2348/69. Hence, the supernatant was diluted at the various volumes shown in FIG. 1, inoculated at 10² CFU/ml into serum-SAPI media, and incubated statically for 17 hrs at 37° C. in a humidified CO₂ incubator. Growth levels were measured using Luria agar and pour plate analysis. The values shown are means of duplicate plate counts; variation between individual assays was no more than 5%.

Referring to FIG. 1, there is shown a histogram illustrating the growth response (CFU/ml) against a range of dilutions of E. coli E2348/69 strains, wild-type (Wt), enterobactin over-producer fes mutant (Ent o/p) and enterobactin non-producer entA mutant (Ent n/p). The Figure shows that there is a growth-stimulating activity (or compound) present in the filter-sterilised culture supernatants of wild-type E. coli E2348/69 grown for 18 hrs in M9 media supplemented with 0.4% (v/v) glucose. FIG. 1 also shows comparative analyses of a growth-inducing activity produced in similar formulation M9 culture supernatants of the E2348/69 enterobactin over-producing mutant (fes) and a mutant deficient in enterobactin synthesis (entA). The Figure shows that the enterobactin non-producing strain (entA) supernatants contain little or no activity, while considerably more activity is synthesised by the enterobactin over-producer (fes) strain than by the wild-type strain.

### Example 2

### Test the Stability of Growth Inducer from E. coli in M9 Media

The inventors wanted to examine the stability of the growth inducer compound that was produced by E. coli in M9. The inventors therefore re-assayed the supernatants from the wild-type and enterobactin over-producer (o/p) cultures produced in Example 1, after 24 hrs storage at 4° C. The values shown are means of duplicate plate counts, and variation between individual assays was no more than 5%. FIG. 2 shows that most of the activity produced by the wild-type strain had decayed within only a single day of storage. FIG. 2 also shows that the activity produced by the E2348/69 enterobactin over-producer fes mutant (o/p) is as unstable to storage as the activity produced by the wild-type strain. The activities in the culture supernatants of wild-type E2348/69 and the fes mutant were also fully functional in siderophore-cross feeding assays (data not shown).

Although the inventors do not wish to be bound by any hypothesis, they believe that the data in FIGS. 1 and 2 suggest that a functional enterobactin synthesis pathway is needed for the production of the growth stimulator in supernatants from the wild-type strain of E2348/69. Moreover, considered collectively, they believe that this data also suggests that the activity produced by the strains previously used for Bacxell™ (NE−AI) synthesis, ie E. coli O157:H7 and E. coli E2348/69, when cultured in M9 media supplemented with 0.4% (v/v) glucose was in fact the siderophore enterobactin, a secreted low molecular weight iron-scavenging molecule which is highly unstable once synthesised.

A limited series of similar analyses to those described in Examples 1 and 2 were also performed with E. coli O157:H7. Very similar results to those shown in FIGS. 1 and 2 were also obtained with E. coli O157:H7 wildtype, and fes and entA mutants (data not shown).

The inventors also investigated whether or not Salmonella spp. were able to produce any growth activity in M9 and using strain SL1344. As shown in FIG. 3, Salmonella strain SL1344 exhibited even less activity that E. coli. The inventors found that about 50 times less activity was produced by SL 1344 in M9 media using the assay methodology described in FIG. 2 (data not shown). This suggests that Salmonella spp. would also be unsuitable for the production of a growth inducer in minimal media, such as M9.

Accordingly, the data in FIGS. 1, 2 and 3 show that the strains that were previously employed for Bacxell™ (NE−AI) synthesis were highly unsuitable for producing a stable growth inducer/stimulator molecule in non-complex minimal media, such as M9. This prompted the inventors to search for a bacterial species that was able to synthesise in an economical minimal media (such as M9) a bacterial growth-stimulating inducer compound that had the functional properties of the Bacxell™, but which was also stable to storage.

### Example 3

### Production of a Stable Growth Inducer from H. alvei in M9 Media

As part of a separate research project investigating iron-regulated gene expression in the bacterial species H. alvei (Escherichia albertii), a panel of mutants had been produced that were defective in genes that were thought likely to be influenced by environmental iron levels. These mutants, as well as various wildtype E. albertii strains, were screened for the production of growth-stimulating activities in minimal media M9.

The inventors used one of the 7 diarrhoeagenic Hafnia alvei strains isolated by Albert et al (Albert et al., 1991, Infection and Immunity 59:1507-1513; Albert et al., 1992, Journal of Medical Microbiology 37:310-314). The recent taxonomic reclassification of diarrhoeagenic Hafnia alvei based upon 16S ribosomal RNA gene sequencing and phenotypic analyses has led to the announcement the H. alvei may also be classified as Escherichia albertii.

Referring to FIG. 6, there is shown a sequence alignment of the Leicester “H. alvei” (Escherichia albertii; LEICS, SEQ ID No:1) used in the methods of the invention, the published Australian eae⁺ “H. alvei” (Escherichia albertii; AUS; SEQ ID No:2), a Hafnia alvei type strain (HALVEI; SEQ ID No:3), and partial 16S rRNA sequences from E. coli (RRSA-SEQ ID No:4; RRSB-SEQ ID No:5; RRSC-SEQ ID No:6; RRSD-SEQ ID No:7; RRSE-SEQ ID No:8; RRSG-SEQ ID No:9; RRSH-SEQ ID No:10).

It can be seen that the sequences of the H. alvei strain used by the inventors (LEICS, SEQ ID No.1) and the H. alvei strain of Albert et al. (Australian eae⁺ strain, denoted AUS in FIG. 6, Genbank Accession number Z83203, SEQ ID No. 2) are 100% identical to each other, except for a 14 bp stretch (residues 80-95 indicated by Ns) where the DNA sequencer failed to resolve the sequence. Clearly, both sequences contain three distinguishing nucleotide differences (indicated as white letters on black background at residues 154, 227, and 275), which are not present in any of the seven E. coli 16S rRNA gene sequences that were tested. Nucleotide 154 in the alignment is a cytosine, nucleotide 227 is an adenine, and nucleotide 275 is an adenine. Hence, the inventors have confirmed that the LEICS sequence of H. alvei used in the method of the invention is identical to the original 16S rRNA sequence submitted to Genbank by Albert (Accession numbers Z47360, Z83203, ie AUS) confirming the identity and origin of the strain used by the inventor (Janda, J. M. et al., 1999, Journal of Clinical Microbiology 37: 2399-2401; Ridell, J. et al., 1995, Journal of Clinical Microbiology 33: 2372-2376).

H. alvei used in methods according to the invention were obtained from Dr Stuart Knutton, Birmingham Children's Hospital, Birmingham, UK who himself had obtained the strain from John Albert's lab referred to herein. H. alvei was inoculated at 2×10⁶ CFU/ml into M9 minimal media that had been supplemented with 0.4% (v/v) glucose. The culture was incubated at 37° C. in a rotatory shaking incubator for 18 hrs (final cell density 1.76 OD600 nm, 3.26×10⁸ CFU/ml). Bacteria were removed by centrifugation at 10,000 g for 15 minutes, and the resulting culture supernatant was sterilised by passage through a 0.2 micron stericup filter. The sterile supernatant was assayed at the volumes shown in the figure using test strain E. coli E2348/69 inoculated at 10² CFU/ml into serum-SAPI media, and incubated statically for 17 hrs at 37° C. in a humidified CO₂ incubator. Growth levels were measured using Luria agar and pour plate analysis.

Analysis of the supernatants of cultures of H. alvei grown in minimal M9 media using the methodology described in relation to the results shown in FIGS. 1 and 2, led to the surprising discovery of a bacterial strain that, when grown in M9 media, produced not only a highly potent growth-stimulating compound, but one which was also significantly stable to storage.

Referring to FIG. 4, there is shown the growth-stimulating activity in the H. alvei culture at day 0 and after 5 days storage at 4° C. The H. alvei supernatants contain more than 5-fold more activity than the equivalent wild-type E. coli and Salmonella M9 supernatants shown in FIG. 1. The data also indicate that the activity in the H. alvei supernatant is surprisingly stable to storage, unlike the activity synthesised in M9 under similar conditions by E. coli as shown in FIG. 2. Variation between individual assays in FIG. 4 was no more than 5%.

Since the bacterial growth-inducing characteristics are similar to those described for Bacxell™, the inventors characterised the growth-inducing compound of the H. alvei culture in order to determine if they had isolated a novel growth inducer, or whether this newly identified molecular species was merely the NE−AI (Bacxell™) disclosed previously. For identification purposes, the inventors termed the growth inducer compound that is produced by H. alvei in M9 media as “BuGro™”.

### Example 4

### Characteristics of the Biosynthesis of BuGro™

The most significant difference between the microbial synthesis of the Bacxell™ NE−AI autoinducer described in WO 98/53047 and BuGro™ is that the former is only produced in serum-supplemented complex SAPI media with production of the compound being dependent on addition of a catecholamine such as noradrenaline (norepinephrine, NE) or the autoinducer itself (the NE−AI is by definition an inducer of its own synthesis). However, in marked contrast, the BuGro™ activity is produced constitutively when H. alvei is cultured in minimal media, such as M9, and is not induced by the addition of NE.

The inventors set out to demonstrate the effect of adding norepinephrine (NE) to the biosynthesis of BuGro™ in M9. Therefore, H. alvei was inoculated at 2×10⁶ CFU/ml into M9 minimal media supplemented with 0.4% (w/v) glucose only (ie no addition, N/A), or with 5% culture supernatant from a previous H. alvei M9 culture (approximately 50 units/ml) (HA), or with 100 μM norepinephrine (NE). The cultures were incubated at 37° C. in a rotatory shaking incubator for 18 hrs (final cell density: 2.17, 2.32 and 2.39×10⁸ CFU/ml). Bacteria were removed by centrifugation at 10,000 g for 15 minutes, and the resulting culture supernatant was sterilised by passage through a 0.2 micron stericup filter. The sterile supernatant was assayed at various volumes using test strain E. coli E2348/69 inoculated at 10² CFU/ml into serum-SAPI media and incubated statically for 17 hrs at 37° C. in a humidified CO₂ incubator. Growth levels were measured using Luria agar and pour plate analysis.

Referring to FIG. 5, there is shown the growth response of the E. coli test strain when incubated with the growth-stimulating activity produced by H. alveii. FIG. 5 surprisingly shows that the synthesis of the H. alvei activity (ie of the BuGro™ compound) is significantly repressed if culture supernatants containing the BuGro™ activity are added at the inoculum stage to M9 cultures of H. alveii. Accordingly, BuGro™ cannot be an autoinducer. Indeed, addition of the growth stimulating activity to the HA culture significantly reduced subsequent synthesis of the activity, which is in marked contrast to the Bacxell™ NE−AI, which can induce its own synthesis.

Accordingly, the inventors believe that they had isolated a novel bacterial growth-inducing compound (BuGro™) by growing H. alvei in M9 minimal media, which is able to induce the growth of bacterial cells and so can be described as an efficient bacterial growth inducer, which exhibits surprising stability to storage, but which was not an autoinducer of its own synthesis. This is in contrast to the Bacxell™ compound which is made by growing E. coli in complex SAPI media, and which is an autoinducer.

### Example 5

### Purification of BuGro™ and Physical Characteristics of its Microbiologically Active Molecular Species

The isolation and purification method that is used for the Bacxell™ NE−AI as detailed in WO 98/53047 cannot be applied to the isolation and purification of BuGro™ because of the difference in the culture media used for their synthesis, ie. Bacxell™ is produced in complex serum-supplemented SAPI media, whereas BuGro™ is produced in minimal M9 media. However, because Bacxell™ and BuGro™ are both low molecular weight electronegative compounds, there are at least some similarities in terms of the types of chromatographic technique and media that may be used for their purification. The culture conditions, extraction procedures, and purification of the BuGro™ compound are described below.

### (i) Culture Conditions and Synthesis of BuGro™

An overnight inoculum of H. alvei (E. albertii) grown up in M9 media containing 0.4% (w/v) glucose was aseptically washed to remove residual BuGro™, re-suspended to the same volume in fresh M9 media, and used to inoculate at a rate of 5 ml of inoculum/1 litre of M9 supplemented with 0.4% (v/v) glucose (approximately 10⁵-10⁶ CFU/ml). This culture was incubated overnight (typically around 17-24 hrs) at 37° C. in an air shaking incubator set at 180 rpm. Typical optical densities of about 1.5-2.0 at 600 nm may be used, which is equivalent to about 2-3×10⁸ CFU/ml.

(ii) Extraction with PBE Resin and TEAB (triethylammonium bicarbonate) Buffer

Bacteria were removed by centrifugation of the M9 culture for 20 min at 7000 g. The supernatant was decanted into clean glass ware, filter sterilised using a 1 litre 0.22 μM Millipore stericup filter, and transferred into a clean, sterile 2-litre flask. Either in a cold room, or at room temperature with the flask sitting in a box of ice, 25 ml of PBE anion exchange resin was added per 1 litre of supernatant, along with TEAB buffer to a final concentration of 10 mM. The resin was stirred for 2-3 hrs, at a rotational velocity of around 60-80 rpm. The resin-bound BuGro™ was collected by Millipore stericup filtration, and residual BuGro™ activity in the supernatant was re-extracted with more PBE resin as described above.

To elute the BuGro™ from the PBE resin, 100 ml of 1 M TEAB was added per 25 ml of resin-BuGro™ and the slurry stirred for 2 hrs at 0-4° C. The eluted activity was collected by re-filtration of the resin through a 0.22 μM Millipore Stericup filter, retaining the supernatant (which contained the eluted BuGro™ compound) and the resin-BuGro™. The resin-BuGro™ was then re-extracted at least 5 more times with a similar volume of 1 M TEAB. Eluates were pooled, and diluted 1:3 with nanopure quality water to enable freezing and lyophilised until dryness. Freeze-dried BuGro™ can be re-constituted in most biologically compatible buffers, provided the pH of the buffer is about neutral (this is needed for long-term storage stability).

A quantitative description of the recovery of activity from each of the processing stages of a typical BuGro™ preparation is shown in Table 1.

TABLE 1
Recovery of activity levels from a typical BuGro ™ preparation.

| | | | Proportion |
|---|---|---|---|
| | | Total | of initial |
| Stage | Volume/ml | Activity | activity, % |
| Initial activity in M9 culture | 4 000 | 1,320,000 | 100 |
| supernatant | | | |
| Extraction 1-5 | 500 | 520,000 | 39.4 |
| Non-adsorbed activity 1 | 4 000 | 640,000 | 48.5 |
| Extraction 2, 1-5 | 500 | 287,000 | 21.7 |
| Non-adsorbed activity 2 | 4 000 | 80,000 | 6.1 |
| Total adsorbed Activity | N/a | 927,000 | 61.1 |

In general, around 60-65% of the total activity of the BuGro™ preparation (initial activity levels of around 0.3-0.5 M units per litre are typical) is recoverable by extraction with PBE resin and elution with TEAB buffer. The BuGro™ residual activity remains either in the twice PBE resin-extracted M9 supernatant, or in a form which is very tightly adsorbed to the PBE resin. Further elution with TEAB buffer beyond the 5^{th} extraction of the PBE resin has been found to be non-cost effective in terms of reagents and manpower. Hence, Table 1 shows the recovery of BuGro™ activity levels from different steps of the extraction and resin elution protocol. The whole procedure on average takes 1.5-2 working days to complete, which significantly contrasts with 5-9 working days for Bacxell™ isolation. This demonstrates the clear advantage of producing BuGro™ in M9 compared to Bacxell™ in complex serum-based media.

(iii) Definition of a BuGro™ Unit of Activity

A “Unit of Activity” is the quantity of BuGro™ required to stimulate the growth of test strain E. coli E2348/69 from an initial inoculum cell density of approximately 10² CFU/ml to 10⁷ CFU/ml, under the following culture conditions: 17 hours static growth at 37° C. in a 5% CO₂/air humidified incubator in SAPI minimal media supplemented with 30% (v/v) bovine serum, (Lyte et al 1996, FEMS Microbiol Lett 139:155-159; Freestone et al, 1999, FEMS Microbiol. Lett. 172: 53-60).

### (iv) Further Purification and Structural Characterisation of BuGro™

For structural analysis predominantly using mass spectroscopy, crude BuGro™ was re-constituted in a 20-fold concentrated form (relative to un-concentrated M9 culture supernatant) in 0.20 M TEAB buffer, and fractionated initially by gel filtration chromatography using a Pharmacia Superdex pep HR30 column, equilibrated in 0.2 M TEAB (effective column dimensions: 1 cm by 50 cm).

Referring to FIGS. 7a and 7b, there are shown Superdex pep analytical chromatography column fractionation profiles of crude BuGro™. FIG. 7a illustrates a UV chromatogram of a typical Superdex pep column fractionation of concentrated crude BuGro™ (the fractionation profile for about 70,000 units of BuGro™ activity is shown). FIG. 7b shows a growth-stimulating activity profile for 1 μl of the 1 ml fractions shown in FIG. 7a, showing the positions of BuGro™ activity peaks 1, 2 and 3. All three of the BuGro™ peaks shown are heat-stable, electronegative, and capable of resuscitating “not immediately culturable” (NIC), although peaks 2 and 3 are the most abundant and most active (in terms of concentration).

FIGS. 7a and 7b reveal that BuGro™ is a heterogeneous mixture that contains three size-separatable molecular species (Peaks 1, 2 and 3), which all possess bacterial growth-stimulating activity. Calibration of the gel filtration column shown in FIG. 7b with standard proteins of known molecular weight allowed estimates to be made of the relative molecular weight of the 3 BuGro™ activity species. Peak 1 is 5 kDa or greater, Peak 2 is about 1100-1200 Da eluting at the same volume as vitamin B12, which is 1200 Da. Peak 3 is about 800-1000 Da, but is believed to have a tendency to polymerise to higher molecular weight species. It will be appreciated that Peaks 2 and 3 are the most abundant BuGro™ activity species.

Further purification of the three BuGro™ activity peaks shown in FIG. 7b was obtained using anion exchange chromatography on a MonoP chromatofocussing column combined with size-separation using a Superdex pep gel filtration column, with details as described below.

### (v) Crude BuGro™ (Labeled as “12 (Crude)” on the MS Spectra in FIG. 8)

The crude BuGro™ shown in FIG. 8 is the same material and species of activity used to make the chromatogram and activity profile shown in FIG. 7. It should be noted that this material was poorly soluble in 100% ethanol, but highly soluble in more aqueous solvents. Also, when acidified, the crude BuGro™ changed in colour from orange-peach to yellow, regaining its original colour on re-neutralisation. All the three peaks are heat-stable, ie they can be boiled for an hour without significant loss of activity, and are each capable of resuscitating highly stressed/non-culturable bacteria. However, unlike the autoinducer Bacxell™ (NE−AI), all 3 BuGro™ peaks possess siderophore activity, although possessed stability/other properties that were atypical of this class of low molecular weight Fe-chelators (see FIG. 12, and Freestone et al, 2003, FEMS Microbiol. Lett. 222: 39-43).

### (vi) Peak 1 (not Shown in FIG. 8)

Peak 1 is variable in terms of the amount that is synthesized, and it is on occasion, difficult to detect. In colour, it is peach-orange. In terms of its relative elution on gel filtration columns, Peak 1 has the highest molecular mass of the three peaks, and based, on its elution volume, has an apparent molecular weight of over 5 kDa. It is the less electronegative than peaks 2 and 3, eluting from a Mono P column at about 40% B, ie about 0.40 M TEAB.

(vii) Peak 2 (Rows 3 and 1 on the MS Spectra Shown in FIG. 8)

Peak 2 fractionates from a Superdex pep gel filtration column as a much more complex mixture which requires substantial further purification. This is achieved using a weak anion exchange (chromatofocussing) column (Pharmacia Mono P 5/5) and a TEAB buffer gradient of 0-1.0 M. On further analysis of the peak 2 of BuGro™ pool by Mono P chromatography, it becomes clear that there are two distinct and separatable species in the single peak of activity identified in the peak 2 gel filtration column fractions shown in FIG. 7. These two species (denoted 2/1 and 2/2) differ in degree of negative charge, but not in size. Peaks 2/1 and 2/2 refer to the order in which they elute from the column using a 0-1 M TEAB gradient. Peak 2/1 elutes at about 55-65% B, ie about 0.6 M TEAB, whereas Peak 2/2 elutes much later in the gradient, around 85-95% B, ie about 0.9 M TEAB. Both are wine-pink in colour, and are extremely stable to further chromatographic processing and storage.

(viii) Peak 3 (Row 5 on the MS Spectra Shown in FIG. 8)

Peak 3 can be purified to a level approaching homogeneity using multiple rounds of Superdex pep gel filtration chromatography alone. Peak 3 seems to have a similar elution volume to Vitamin B12, which suggests that its molecular weight is greater than 1000 Da. Although this peak elutes discretely over a small volume (typically 2-3 ml), it seems to have a tendency to polymerise when lyophilized. Peak 3 fractions are reconstituted at high concentration (10-20 times higher than would be obtained when using a crude BuGro™ fractionation as shown in FIG. 7), as well as shifting in visible wavelength characteristics to a yellow colour. When re-analysed by gel filtration chromatograph as shown in FIG. 7, the polymerised peak 3 is often found to have undergone a shift in elution volume, from around 30-32 ml to around 22-26 ml, ie the elution volume of Peak 2. This higher molecular weight polymerised Peak 3 species has usually also lost most of its growth-stimulating activity. Peak 3 can also be purified using Mono P anion exchange column fractionation, where it elutes at around 50-60% B, ie 0.55 M TEAB.

### Example 6

### Mass Spectroscopy Analysis of BuGro™ Activity Peaks

Peak 1 was not included in the MS analyses shown in FIG. 8 due to it being in too low abundance and technical difficulties in separating this species of activity from co-eluting contaminants. Peaks 2 and 3 are the major species in terms of quantity and growth-stimulation potency in the crude BuGro™ concentrate, and so these were purified to as high a level of homogeneity as possible, and subjected to MS analysis as described below.

### (i) Crude BuGro™ Concentrate

These spectra are complex, which is consistent with the fact this is a culture supernatant concentrate that has only been slightly purified by anion exchange adsorption and elution. The crude BuGro™ used for the spectrum in FIG. 8 is the same material and amount of activity used to make the chromatogram and activity profile shown in FIG. 7.

Referring to FIGS. 8 and 9, there are shown positive and negative ion mode spectra, respectively, for crude BuGro™ and Peaks 2 and 3. For both FIGS. 8 and 9, the BuGro™ samples were dissolved in 50% aqueous methanol (200 μL). ESI MS in the positive detection mode was carried out using formic acid (1%) (1:1) as the proton donor, and in the negative mode with no additions.

### (ii) Peak 2 and Peak 3

The Peak 2 and 3 species are the most abundant activity species in BuGro™ in terms of relative amounts. As previously stated, Peak 2 exists in two electronegative ionisation states, 2/1 and 2/2. Peak 3 is the lowest molecular weight BuGro™ activity species, and the most discrete in terms of its fractionation characteristics. At high concentration, Peak 3 has a tendency to polymerise, acquiring a Vₑ on gel filtration columns similar to that of peak 2, suggesting that it may be a breakdown product/subunit of Peak 2. FIG. 8 shows that there are few molecular species higher in mass than 1200 Da visible in the spectra of Peak 2/1 and 2/2, which is consistent with a 1100-1200 Da estimate of the molecular weight of these two peaks made by gel filtration chromatography. The spectrum for Peak 3 has similar species in it to the Peak 2 set of spectra, which again would be consistent with Peak 3 being a component or subunit of Peak 2. It should be noted that the spectra shown in FIG. 8 are all negatively charged, which is consistent with the chromatographic properties of the BuGro™ peaks. Significant information regarding the inter-relationship of Peak 2 and Peak 3 comes from molecular fragmentation analysis of Peaks 2/1 and 2/2 and their degradation products.

Referring to FIGS. 10 and 11, there are shown the results of ESI-MSMS fragmentation analysis of the prominent Peak 2/1 and 2/2 peaks in the negative ion mode of FIG. 9. In both cases, fragmentation analysis was carried out using argon as the collision gas at 15 psi and collision energy of 20 eV (typically). Spectra were obtained from both Peak 2 peaks which have a high level of similarity in their patterns of fragmentation. The highest mass peak that could be discerned from the BuGro™ peak 2 spectra was 1167 in the negative ion mode, providing further support for the hypothesis that Peak 2 at least is derived from a parent molecule of mass greater than 1100 Da. The 1167 species breaks down to 1069, which in turn breaks down to 971, the differences of 98 mass units suggesting that these may be phosphate moieties. While the inventors do not wish to be bound by any hypothesis, they believe these moieties may be two terminal phosphates. Other instances being 1047 fragmenting to 949, 583 to 485, and in the positive ion mode 951 (953) breaks down to 857, all suggesting that phosphate may be part of the structure. In the negative ion mode, peaks with m/z 927, 949, 485, 463, 240, 178 all seem to be prominent and related (927 gives rise to 463, and in turn to 240 and 178). Correspondingly, 951, 487 and 242 (adding two more mass units to 949, 485 and 240, respectively) appear in the positive ion mode, although interestingly the m/z 180 species does not (suggesting that perhaps the source of this is not easily protonated, possibly indicating that the 179 species is a catechol derivative with an acid group). It should be appreciated that these are m/z values and not ‘m’ values, so charge states of the molecules are also important.

The precise chemical structure of Peak 2 and 3 species and their fragmented components remains to be determined, although the masses observed for the parent Peak 2 species and the spectra of these molecules generally are very unlike those reported in WO 98/53047 for the NE−AI autoinducer, Bacxell™ Further analysis of the BuGro™ compound by NMR spectroscopy using highly purified Peak 3 and D₂O as solvent revealed the presence of aromatic chemical group(s) within this molecule, and interestingly, a structural motif very similar to that of the amino acid serine.

### Example 7

### Properties of BuGro™—Demonstration that BuGro™ and Peak 1-3 Preparations Possess Siderophore Activity

Previously, the inventors have shown that the siderophore enterobactin is an integral molecule in the mechanism by which catecholamines, such as NE, can induce growth of enteric bacteria such as E. coli (Freestone et al, 2000, J. Bact. 182: 6091-6098; Freestone et al, 2003, FEMS Microbiol. Lett. 222: 39-43). Furthermore, the inventors have also clearly demonstrated that the Bacxell™ autoinducer produced by growing E. coli in SAPI media does not exhibit siderophore activity.

The inventors wanted to investigate whether or not BuGro™ exhibited siderophore activity, and carried out an experiment using the methodology described in Freestone et al (2003, FEMS Microbiol. Lett. 222: 39-43). An enterobactin non-producer entA mutant was first incubated in buffered minimal media supplemented with 0.2% (w/v) glucose plus 2×10⁵ CPM/ml of ⁵⁵Fe-labelled transferrin that was contained within dialysis tubing that was permeable to small molecules of molecular weight of less than 5000 Da, plus the compounds described in the legend to FIG. 12. After 6 hrs, the bacteria were harvested, washed and measured for ⁵⁵Fe-uptake using scintillation counting as described in Freestone et al (2003, FEMS Microbiol. Lett. 222: 39-43).

Referring to FIG. 12, there is shown a histogram illustrating the ability of crude BuGro and purified Peaks 1, 2 and 3 to enable 2×10⁸CFU/ml of an entA enterobactin-deficient strain of E. coli E2348/69 to acquire iron (in the form of ⁵⁵Fe) from ⁵⁵Fe-labelled transferrin. Hence, FIG. 12 shows that the BuGro™ peaks were able to substitute for the siderophore enterobactin in enabling a non-siderophore synthesising E. coli entA mutant to acquire iron from norepinephrine-transferrin complexes. Accordingly, surprisingly, the inventors have found that all three of the BuGro™ peaks each possess siderophore-activity similar to that of enterobactin. It is important to note that the NE−AI, Bacxell™, does not possess such siderophore activity, even when supplied at a level of 20-times more than would be required to induce growth in serum or blood-supplemented media. This indicates that the BuGro™ compound is functionally distinct from the Bacxell™ compound, even though both compounds are able to function as bacterial growth stimulators and resuscitators. It should be noted that the siderophore enterobactin is highly unstable once synthesised in minimal media such as M9, as was observed in FIG. 2. Furthermore BuGro™ cannot be enterobactin due to its other properties and its molecular weight.

### Example 8

### BuGro™ Stability

BuGro™ and the individual activity Peaks 1, 2 and 3 are each stable to 60 minutes boiling when in a heat-stable buffer such as 50 mM Tris-HCl, pH 7.5 or phosphate buffered saline (PBS). All are fully stable to lyophilisation, and if maintained in a lyophilised form, crude BuGro™ is 90-95% stable to storage at 4° C. for at least 2 years. Hence, clearly BuGro™ is very stable to storage and to heat-treatment. Further analysis of the properties of this compound also revealed that it insensitive to enzymatic inactivation by ribonuclease, deoxyribonuclease, proteases such as proteinase K and trypsin, and lipase (data not shown).

### Example 9

### Comparison of the Characteristics of BuGro™ and Bacxell™

A comparison of the properties of BuGro™ according to the present invention and Bacxell™ (the autoinducer disclosed in WO 98/53047) can be seen in Table 2.

TABLE 2
Comparison of BuGro ™ and Bacxell ™

| | Criteria | |
|---|---|---|
| BUGRO | Comment | BACXELL |
| | Chemical | |
| | characteristics | |
| 1. Electronegative (4 charged forms), | Broadly Similar | 1. Electronegative (2 charged forms), |
| polar, hydrophilic. | | polar, hydrophilic. |
| 2. N/D | | 2. Binds metals. |
| 3. Highly water soluble. | | 3. Highly water soluble. |
| | Stability | |
| 1. Stable to heating, lyophilization and | Broadly Similar | 1. Stable to heating, lyophilization and |
| prolonged storage. | | moderate storage. |
| 2. Stable to ribonuclease, | | 2. Stable to ribonuclease, |
| deoxyribonuclease, protease, | | deoxyribonuclease, protease, |
| phosphatase, phosphodiesterase | | phosphatase, phosphodiesterase |
| digestion. | | digestion. |
| 3. Unstable to acidic pH, more stable to | | 3. Unstable to both pH extremes. |
| high pH. | | |
| | Structural | |
| | characteristics | |
| 1. Catecholate compound containing | 1. Similar | 1. Aromatic group present, most |
| serine, possibly trimeric. | | probably catecholate in structure. Most |
| | | likely monomeric/dimeric. |
| 2. Molecular weight around ~1100 Da | 2. Different | 2. Approximately 500-600 Da. |
| No higher molecular weight form. | | Also a ~10 kDa serum-protein bound |
| | | form. |
| 3. All 3 species of Bugro activity are | 3. Different | 3. Bacxell non-functional in |
| functional in siderophore assays. | | siderophore assays. |
| | Synthesis | |
| 1. Synthesised by Hafnia in minimal | 1. Different | 1. Synthesised by E. coli in serum- |
| medium only | | based media. E. coli does not make |
| | | BuGro in minimal medium. |
| 2. Enterobactin genes involved in | 2. Similar | 2. Enterobactin genes involved in |
| synthesis | | synthesis |
| 3. Hafnia makes a growth stimulating | 3. Different | 3. E. coli does not make BuGro-like |
| activity in serum-media, but this is | | activity in minimal media |
| chromatographically unlike both the E. coli | | |
| Bacxell, and M9-made Hafnia BuGro. | | |
| 4. Addition of BuGro to cultures of Hafnia | 4. Different | 4. Induced by norepinephrine, but also |
| alvei in M9 media represses BuGro | | Bacxell induces own synthesis in |
| synthesis. No effect of norepinephrine on | | serum-based media. |
| BuGro synthesis. | | |
| 5. Significantly repressed under conditions | 5. Different | 5. Not substantially repressed by iron |
| of iron excess | | excess. |
| | Purification | |
| Anion exchange, analytical scale gel | Different order | Preparative Superdex pep gel |
| filtration chromatography, | and columns | filtration, anion exchange, analytical |
| | | gel filtration |
| | Biological | |
| | Effects | |
| 1. Stimulates bacterial growth and | All Similar | 1. Stimulates bacterial growth and |
| virulence factor expression. | | virulence factor expression. |
| 2. Revives viable but non-culturable and | | 2. Revives viable but non-culturable |
| heat/other stressed bacteria. | | and heat/other stressed bacteria. |
| 3. Very broad cross-species activity | | 3. Very broad cross-species activity |

While it is clear that the biological (growth enhancement and bacterial resuscitation) properties of the Bacxell™ and BuGro™ are alike, and it is believed that similar genes may be involved in their synthesis, other aspects such as molecular weight differences and the ability to act as a siderophore indicate that they are not at all the same compound.

It should be appreciated that BuGro™ has a role to play in microbiological process where enhancement of bacterial growth is desirable. For example, BuGro™ has applications in microbial fermentation technologies (eg production of primary and secondary metabolites), in culture media and diagnostics, and in environmental monitoring. The following illustrates some of the ways in which BuGro™ may be used to enhance existing diagnostic systems, as well as more novel applications, such as bio-prospecting from natural resources, such as soil.

### Example 10

### Microbiological Properties of BuGro™ Relevant to Bacterial Diagnostics (Blood Samples)

The inventors wanted to test the efficacy of BuGro™ to act as a bacterial growth inducer, which may be used in the analyses of microbial populations in biological samples, such as blood.

Hence, ten E. coli cells were inoculated into 10 ml BacT/ALERT media (a commercially available, clinical microbiological blood culture media widely used in UK and European hospitals) supplemented with 30% blood (the recommended volume for this volume of BacT/ALERT media) and then further supplemented with either no addition of BuGro™ (N/A) or 50 units/ml BuGro™ (HA). The cultures were then incubated for 14 hours at 37° C. Growth levels were determined by serial dilution of the cultures and plate counting using Luria agar.

Referring to FIG. 13, there is shown the growth levels of the ten E. coli cells in the blood sample with either no additions (N/A) or 50 units/ml BuGro (HA). As can be seen, BuGro™ is able to significantly enhance the growth of the E. coli in blood compared to the growth recovery of E. coli in the absence of BuGro™. In fact, the growth induction/recovery rate is at least 4 orders of magnitude greater in the presence of BuGro™ than if it were not added.

### Example 11

### Microbiological Properties of BuGro™ Relevant to Bacterial Diagnostics (Antibiotic-Damaged Bacteria)

The inventors also wanted to investigate the ability of BuGro™ to induce, revive or stimulate growth of not-immediately-culturable (NIC) bacteria present in a sample containing the antibiotic ampicillin (to reflect a clinical situation in which infectious blood-borne bacteria within a patient have been treated with an antibiotic). Hence, 10⁸ CFU/ml ampicillin-treated E. coli cells were serially diluted into BacT/ALERT media (no blood control) supplemented with either no additions (N/A) or 50 units/ml BuGro™ (HA), and incubated for 14 hours at 37° C. Growth levels were determined by serial dilution of the cultures and plate counting using Luria agar.

Referring to FIG. 14, there are shown the growth levels of 10⁸ CFU/ml ampicillin-treated E. coli cells in BacT/ALERT media supplemented with either no addition (N/A) or addition of 50 units/ml BuGro™ (HA). The “Control” bar refers to the growth of non-antibiotic-treated bacteria with no additions, which are fully viable and therefore capable of un-impeded growth to 10⁸ CFU/ml in rich media, such as BacT/ALERT. As can be seen in FIG. 14, in the presence of BuGro™, an increase in bacterial growth of nearly 2 logs was observed, indicating that BuGro™ is able to facilitate the activation of growth in antibiotic-stressed bacteria. This has obvious implications for use of BuGro™ in shortening the time required for the detection of antibiotic-damaged bacteria in clinical microbiology culture diagnostics samples.

In summary, BuGro™ is able to enhance the recovery in blood and serum of antibiotic-damaged and apparently non-culturable bacteria, as shown in FIGS. 13 and 14, demonstrating a clear application for BuGro™ in bacterial clinical and environmental diagnostic technologies. Hence, it is evident that BuGro™ shares the same ability of the NE−AI, Bacxell™, to induce bacterial growth in iron-restricted mammalian tissue fluids such as blood or serum, but is an improvement due to its increased stability.

### Example 12

### Utility of BuGro™ in Blood Culture Diagnostics

One particularly valuable property of BuGro™ is its ability to facilitate recovery of pathogenic bacteria that have experienced damage by antibiotics to the extent that they are non-culturable. This is a useful discovery, given that current microbiology blood culture diagnostics are poor at detecting bacteria (a rate of negative culture diagnoses of greater than 90%), even when the patient's physical status is indicative of a blood-associated infection.

Bacterial innocula (blood stream isolates of E. coli) were prepared by dilution (1:50) of overnight cultures (grown in a 37° C. shaking incubator) into appropriate media (BacTALERT alone or supplemented 30% (v/v) with fresh venous blood) and further cultured as specified above, until growth was approximately logarithmic. Bacteria were divided into 2 portions, one of which contained an antibiotic of choice (kanamycin, trimethoprim, or chloramphenicol), at a concentration of at least 6 times the minimum inhibitory concentration, MIC. These cultures were grown for a further 2-3 hours. The viable plate count was determined for each culture, as was, where appropriate, the OD₆₀₀. Control and antibiotic-stressed cultures were then serially diluted in 1:10 steps in a 24-well plate to a nominal single cfu/ml into fresh BacTALERT media alone or supplemented 30% (v/v) with fresh venous blood +/−40 units/ml BuGro™. Plates were incubated statically in a humidified incubator set at 37° C. for up to 36 hours. All plates were visually inspected for growth at 14, 21 and 36 hours, and where appropriately scored for viable colonies (where growth was sub-visible) using pour plate analysis. Representative profiles of how BuGro™ enhanced the growth of an E. coli blood stream isolate treated with ampicillin (100 μg/ml) in BacTALERT media alone and supplemented with blood are shown in FIGS. 13 and 14. Controls are the growth profiles of the un-treated culture.

Therefore, in addition to ampicillin-damaged bacteria described in Example 11, the inventors have also shown that BuGro™ can improve the recovery, and therefore decrease the time of detection of bacteria treated with inhibitory concentrations of kanamycin (50 μg/ml), trimethoprim (50 μg/ml), and chloramphenicol (10 μg/ml). The results (not shown) indicate that BuGro™ is a potent bacterial inducer and growth-stimulant that is functional in the Biomerieux BacT/ALERT media either when used alone, or with a blood supplementation similar to that used in clinical diagnostic settings. The inventors envisage that inclusion of BuGro™ into blood culture diagnostic media is likely to maximise detection of low numbers of bacteria in a shorter time frame. The inventors therefore envisage an immediate application in the field of in vitro diagnostics of infectious diseases.

### Example 13

### Utility of BuGro™ in Analyses of the Microbial Composition of Food Samples

The inventors wished to evaluate the utility of BuGro™ at inducing bacterial growth in food samples, such as dried herbs. Dried herbs (parsley, thyme, oregano, sage and marjoram, 0.12 g) from a freshly opened pot were vortexed for 2 minutes with 50 ml of sterile Luria Broth (LB). The suspension was spun at 4000 g for 20 minutes to pellet solid matter, and the liquid component was aseptically removed and divided between 4 sterile polypropylene tubes, two of which contained 10 units/ml BuGro™, or the other two contained an equivalent volume of water, as negative control. The tubes were then incubated statically for 16 hours at 37° C., mixed, and analysed for microbial content by serial dilution and plating onto Luria agar.

The results of a typical analysis are show in FIG. 15. The bacteria predominant in the +BuGro™ herb infusion were producers of copious amounts of exo-polysaccharide, and gave a negative Gram-stain, suggesting it may have been an Erwinia or Klebsiella species. However, given the herbaceous nature of the sample, Xanthomonas was also a possibility, although the species isolated possessed a very potent haemolytic activity when cultured on blood agar. The major species isolated from the −BuGro™ cultures did not possess any haemolytic activity, and strained Gram-positive, suggesting they may have been Bacillus.

The presence of BuGro™ caused a very profound alteration in the species profile of bacteria isolated from the herb samples that were tested. The fact that the major species isolated from the samples in the presence of BuGro™ was haemolytic suggests that it may have posed a health hazard. This is worrying given that the instructions on the packaging of the herb preparation tested indicated that the product could be sprinkled directly onto cooked foods. Accordingly, the inventors have demonstrated the BuGro™ can be successfully used to induce bacterial growth in food samples.

### Example 14

### Utility of BuGro™ in the Analyses of the Microbial Composition of Soil

The inventors also wished to see if BuGro™ may be used to induce bacterial growth in a soil sample. Two methods were used:—

### Methodology 1

1 g of soil was taken from a domestic garden in a rural area and suspended in 100 ml of a 1/1000 dilution of R2A broth (a specialised media for the isolation of heterotrophic soil micro-organisms) containing an anti-fungal agent (5 μg/ml cyclohexamide). R2A media was used in a 1:1000-fold dilution to maximise recovery of very slow-growing/not immediately culturable soil bacteria, and to minimise overgrowth and domination of the microbial community by more metabolically active/faster growing species. The R2A broth/soil mixture was shaken in a 250 ml baffled flask at 25° C., 180 revolutions/minute for 1 hour to facilitate dispersion of large soil particles. The resulting suspension was filtered through a fine nylon mesh to remove stones and other debris, and aseptically divided into 10 ml aliquots in 25 ml sterile polypropylene tubes containing either no additions (control), or supplemented with purified BuGro™ (10 units of BuGro™/ml of suspension). The BuGro™ supplemented and control cultures were incubated for a further 24 hours at 25° C. with shaking as described above. The cultures were then diluted with phosphate buffered saline (PBS) and enumerated by plating onto solid growth media. All such inoculated plates were incubated at 25° C. for up to 48 hours to allow enumeration of slow growing bacteria. Cultures were analysed for differences in total bacterial cell numbers as shown in FIG. 16, as well as for changes in bacterial species diversity (as shown in FIG. 17).

FIG. 16 shows the total number of bacterial colonies (Gram-positive and Gram-negative) isolated from dilute R2A soil broth cultures that had been incubated for 24 hours in the absence or presence of BuGro™, and which were able to grow on non-selective R2A agar. The histogram shows pooled data sets for three independent experiments involving multiple pairs (n) of supplemented and non-supplemented 10 ml soil cultures (#1, n=5; #2, n=2; #3, n=3). Control R2A soil cultures gave recoverable soil bacterial populations of approximately 2×10⁹ cfu/g of soil, while cultures that had been supplemented with BuGro™ showed an approximately 2.5-fold increase in total bacterial numbers. This indicates that, in addition to possessing the ability to stimulate growth of pathogenic bacteria (eg blood or food samples), BuGro™ is also capable of enhancing the growth of bacterial species present within soil.

The histogram shown in FIG. 17 shows the number of bacterial colonies isolated from dilute R2A soil broth cultures that had been incubated for 24 hours in the absence or presence of BuGro™, and that were able to grow on solid R2A agar (1-5), or other selective media (6-8) containing the following additions:
1. R2A agar only (control, no additions)
2. Polymixin B, 5 μg/ml (selective for Gram-negative bacteria);
3. Polymixin B, 5 μg/ml plus 100 μg/ml ampicillin (selective for ampicillin-resistant Gram-negative bacteria);
4. Vancomycin, 50 μg/ml (selective for Gram-positive bacteria);
5. Vancomycin, 50 μg/ml plus 100 μg/ml ampicillin (selective for ampicillin-resistant Gram-positive bacteria);
6. Citrate agar, no antibiotics (selective for Gram-positive or Gram-negative bacteria capable of metabolising citrate as carbon source)
7. MacConkey agar, no antibiotics (distinguishes between lactose-fermenting and non-fermenting Gram-positive or Gram-negative bacteria);
8. Sheep blood agar, no antibiotics (distinguishes between haemolytic and non-haemolytic Gram-positive or Gram-negative bacteria).

FIG. 17 shows the investigations into the ability of BuGro™ to induce changes in species diversity of soil bacteria populations. Although the ideal approach would have been to identify each individual colony isolated, the enormous number of bacterial species identified as normal residents of soil (tentatively estimated at over 4000) made this impossible. Instead, the inventors chose a methodology involving plating on media selective for various species types.

The histogram shows pooled data sets for a typical set of experiments involving multiple pairs (n=2) of supplemented and non-supplemented 10 ml soil cultures. R2A plates supplemented with antibiotics selective for Gram-negatives (polymixin B and ampicillin) showed a 6-fold increase in bacterial cell numbers for BuGro™-treated samples, while R2A plates supplemented antibiotics selective for Gram-positive bacteria showed an approximate 2-fold increase in cell numbers. This suggests that responsiveness to BuGro™ is a predominantly Gram-negative species phenomenon. Analysis of BuGro™-treated R2A cultures on citrate, MacConkey or sheep blood agar plates gave 2-3-fold increases in cell numbers over unsupplemented controls, comparable to the levels of enhancement observed on non-selective R2A media. However, haemolytic colonies were only observed in BuGro™-supplemented cultures.

### Methodology 2

This approach to demonstrate the ability of BuGro™ to affect soil species diversity involves selective pre-enrichment by incubation of soil samples in soil broth media containing antibiotics selective for either Gram-positive or Gram-negative bacteria prior to BuGro™ treatment, and analysis of the resulting changes in growth patterns using a Biolog™ Microplate culture test kit. This is a commercial method of bacterial species identification which uses a prepared 96-well plate containing 95 different carbon sources, plus a control well containing water only. The ability to metabolise a carbon source is indicated by a change in well colour from clear/colourless to purple. Different bacteria produce a different pattern of carbon source metabolism, which is unique to that particular bacterial species (a ‘metabolic fingerprint’). In the current experiments, the Biolog™ method of culture analysis was not used to identify individual soil microbial species, but to detect differences in composition of the soil bacterial community resulting from preferential enhancement of growth of certain bacteria by BuGro™.

A soil suspension inoculum was prepared by adding 0.2 g of the garden loam used in Methodology 1 to 10 ml of SB soil culture broth (as prepared in Methods for studying the Ecology of Soil Micro-organisms, Edited by D. Parkinson, T. R. G. Gray and S. T Williams IBP Handbook No 19) containing an anti-fungal agent (cyclohexamide 5 μg/ml) plus antibiotics selective for either Gram-positive bacteria (vancomycin 50 μg/ml) or Gram-negative bacteria (polymyxin B, 5 μg/ml and ampicillin and 100 μg/ml). The broth suspension was shaken for 15 hours at room temperature to ensure even dispersion of soil particles. The soil culture was then harvested by centrifugation at 4000 g, room temperature, for 10 minutes, washed twice in 10 ml of SB broth to remove any traces of antibiotic, and the cell pellet re-suspended in 20 ml of the same media. This culture, now enriched for either Gram-positive of Gram-negative bacteria, was divided into two portions, one of which was supplemented with 10 units/ml of BuGro™, or an equivalent volume of water. The supplemented cultures were then incubated with shaking at room temperature for a further 15 hours, before centrifugation and washing with phosphate buffered saline, and re-centrifugation as described above. The washed cell pellets were re-suspended in sufficient Microplate inoculating fluid (provided by the Biolog™ Microplate manufacturers) to give an OD₅₉₀ of between 0.34 and 0.39, and 150 μl of this suspension inoculated into each of the 96 wells of the Biolog™ Microplate as recommended in the manufacturer's instructions. Plates were inoculated in triplicate with each culture suspension, and allowed to incubate at room temperature for up to 7 days. Biolog™ Microplate analysis revealed clear differences between the metabolic fingerprints of cultures incubated in the presence and absence of BuGro™ (FIGS. 18 and 19).

FIGS. 18 and 19 show Biolog™ Microplate profiles of demonstrating the effects of adding BuGro™ to soil cultures enriched for either Gram-positive or Gram-negative soil bacterial species. Filled circles indicate an ability to metabolise the carbon source shown. Such changes in the pattern of carbohydrate utilisation indicate changes in the carbohydrate metabolism profile of the species present. Such results could be interpreted in terms of BuGro™ acting as a metabolic inhibitor or activator, affecting the ability of soil species to utilise the carbon sources provided. However, the inventors have realised from the data shown in FIGS. 18 and 19 that the metabolic fingerprints shown are of mixed bacterial cultures and it is unlikely that BuGro™ could exert similar modulatory effects on all of the bacterial species present. In any case, the inventors have demonstrated that BuGro™ can induce both enhancement of growth (FIGS. 15-17) and changes in species diversity of bacteria present within soil samples (FIGS. 18 and 19). In addition, the inventors have also shown that BuGro™ possesses the ability to resuscitate not immediately culturable bacteria. Therefore, the most likely explanation for the differences in the metabolic fingerprints shown in FIGS. 18 and 19, given that similar cell numbers are used, is that BuGro™ is inducing direct changes in the composition of species within the soil microbial population.

In summary, BuGro™ has been shown to possess the potential to resuscitate bacteria that are stressed or damaged by prolonged incubation in water, suggesting applications in diagnosis and in the monitoring of foodstuffs and environmental samples. The results suggest that the growth properties of BuGro™ can be extended to also include Gram-negative and Gram-positive soil bacteria.

1. A method of producing a bacterial growth inducer, characterized in that the method comprises culturing Hafnia spp. in minimal media, and in that the growth inducer is not an autoinducer.

2. A method according to claim 1, wherein the minimal media comprises a glucose-supplemented buffered salt solution.

3. A method according to claim 1, wherein the minimal media comprises M9 minimal media.

4. A method according to claim 1, wherein the minimal media comprises 0.1-2% (w/v) glucose.

5. A method according to claim 1, wherein the Hafnia species is Hafnia alvei.

6. A method according to claim 5, wherein the H. alvei strain comprises a 16S ribosomal RNA sequence substantially comprising SEQ ID No.1.

7. A method according to claim 1, wherein the culturing step comprises incubating the Hafnia spp. in minimal media at a temperature of about 30° C.-37° C., for at least 5 hours.

8. A method according to claim 1, wherein the method comprises a step of isolating the bacterial growth inducer from the Hafnia spp. growth culture.

9. A method according to claim 8, wherein the method comprises collecting a sample from the Hafnia spp. culture containing the bacterial growth inducer.

10. A method according to claim 9, wherein the method comprises isolating the bacterial growth inducer from a supernatant of the sample.

11. A method according to claim 10, wherein the method comprises a step of fractionating the sample, and isolating a fraction that corresponds to molecular weights of approximately 800-5000 Daltons.

12. A method according to claim 11, wherein fractionation is carried out by means of size exclusion gel filtration.

13. A method according to claim 12, wherein size exclusion gel filtration is performed using anion exchange chromatography.

14. A method according to claim 11, wherein the method comprises an additional step of concentrating the sample prior to the fractionation step.

15. A method according to claim 14, wherein concentration is achieved by means of ultrafiltration.

16. A method according to claim 15, wherein the ultrafiltration step comprises use of a molecular weight cut-off that is greater than 1500 Daltons.

17. A bacterial growth inducer, characterized in that the inducer is prepared by, or obtainable by, culturing Hafnia spp. in minimal media, and in that the growth inducer is not an autoinducer.

18. (canceled)

19. A growth inducer according to claim 17, wherein the bacterial growth inducer has a molecular weight of about 800-5000 Daltons.

20. A growth inducer according to claim 19, wherein the growth inducer has a molecular weight of between about 900 and 2000 Daltons.

21. A growth inducer according to claim 17, wherein the growth inducer exhibits siderophore activity.

22. A growth inducer according to claim 17, wherein the bacterial growth inducer is characterized in that:—
(i) it has a molecular weight of about 800-5000 Daltons;
(ii) it exhibits siderophore activity;
(iii) it is fluorescent;
(iv) it is produced constitutively;
(v) addition of the growth inducer to a culture of Hafnia alvei in M9 represses its own synthesis;
(vi) addition of norepinephrine inducer to a culture of Hafnia alvei in M9 does not induce synthesis of the bacterial growth inducer; and/or
(vii) the production of the growth inducer is repressed under conditions of iron excess.

23. A growth inducer according to claim 17, wherein the bacterial growth inducer is capable of inducing the growth of a Gram-positive bacterium.

24. A growth inducer according to claim 23, wherein the bacterial growth inducer is capable of inducing the growth of Firmicutes, such as Bacilli or Clostridia.

25. A growth inducer according to claim 17, wherein the bacterial growth inducer is capable of inducing the growth of a Gram-negative bacterium.

26. A growth inducer according to claim 25, wherein the bacterial growth inducer is capable of inducing the growth of Staphylococcus spp., Streptococci spp., Pseudomonadales spp., Xanthomonas spp. Enterobacteriales spp., Proteus spp., Serratia spp., Pasteurellales spp., or Vibrionales spp.

27. A growth inducer according to claim 26, wherein the bacterial growth inducer is capable of inducing the growth of Escherichia spp., Salmonella spp., Shigella spp., Yersinia spp., Klebsiella spp., or Erwinia spp.

28. A growth inducer according to claim 17, wherein the bacterial growth inducer is capable of inducing growth of not immediately culturable (NIC) bacteria.

29. (canceled)

30. A method of inducing growth of bacteria in a sample, the method comprising contacting a sample containing bacteria with the bacterial growth inducer according to claim 17, and incubating said sample under conditions suitable for inducing growth of the bacteria.

31. A method according to claim 30, wherein the method comprises a detection step for detecting the growing bacteria.

32. A method according to claim 31, wherein the detection step comprises plating the sample onto suitable media.

33. A kit for inducing growth of bacteria in a sample, the kit comprising the bacterial growth inducer according to claim 17, and optionally instructions for use.

34. (canceled)

35. A method according to claim 30, wherein the sample is a blood sample.

36. A kit according to claim 33, wherein the sample is a blood sample.

37. A method according to claim 30, wherein the sample is a food sample.

38. A kit according to claim 33, wherein the sample is a food sample.

39. A method according to claim 30, wherein the sample is a soil sample.

40. A kit according to claim 33, wherein the sample is a soil sample.
